(19) United States
(12) Patent Application Publication
(10) Pub. No.: US 2005/0164167 A1
(43) Pub. Date: Jul. 28, 2005

(54) Method of discovery and development of broad-spectrum antiviral drugs

(76) Inventors: Benjamin A. Buscher, St. Louis, MO (US); Julie Dyall, St. Louis, MO (US); Jennifer I. Jockel-Balsarotti, St. Louis, MO (US); Andrew K. O'Guin, St. Louis, MO (US); Paul D. Olivo, Clayton, MO (US); Robert M. Roth, St. Louis, MO (US); Yi Zhou, Ballwin, MO (US)

Correspondence Address: HARNESS, DICKEY, & PIERCE, P.L.C, 7700 BONHOMME, STE 400, ST. LOUIS, MO, 63105, US

(21) Appl. No.: 10/766,226

(22) Filed: Jan. 28, 2004

(51) Int. Cl. C12Q 1/70; C12Q 1/68

(52) U.S. Cl. 435/5; 435/6

(57) ABSTRACT

Method for identifying a broad-spectrum antiviral lead compound. Also methods for marketing and delivering a broad-spectrum antiviral compound and methods for treating patients with antiviral infections with a broad-spectrum antiviral drug are disclosed.

### FIELD OF INTEREST

The present invention generally relates to methods for discovery and development of a broad-spectrum antiviral drug. More specifically, the invention relates to methods of development and delivery of a broad-spectrum antiviral drug and the treatment of a viral infection using the broad-spectrum antiviral drug.

### BACKGROUND

The process of antiviral drug discovery requires the screening or assaying of various possible compounds for activity against the replication of one or more viruses. Generally, antiviral drug discovery screening is target-based. In one form of target-based screening, a particular biochemical target is chosen which is believed to be associated with one or more characteristics of a particular virus such as replication. Such biochemical targets are encoded by the viral genome and are often specific to that particular virus. Inhibitors of such biochemical targets are usually active only against the virus from which the target is derived. Candidate antiviral compounds can also be screened to determine the level of inhibition of a particular target that may be common to more than one virus, thereby resulting in inhibition of replication of a broad-spectrum of viruses. Such targets are often a cellular enzyme or a receptor that is known or thought to be essential to the process of viral replication. Suspected viral targets include inosine monophosphate dehydrogenase (IMPDH), S-adenosylhomocysteine hydrolase (SAH), and certain cyclin-dependent kinases (CDK). Such target-based screening has resulted in the identification of only a few broad-spectrum antiviral compounds. However, generally these drugs, while exhibiting broad-spectrum antiviral efficacy, are screened and developed only to specific viruses such as hepatitis C (HCV) for which they demonstrate a high level of antiviral efficacy, such as hepatitis C (HCV).

A second screening approach is an unbiased approach where inhibitors of viral replication are sought without a concern or knowledge of a particular target. An unbiased approach generally involves use of cell cultures because, as obligate intracellular pathogens, viruses only replicate within a cell. Although cell-based screening has been used successfully in other drug-discovery fields, it is difficult to utilize in screening antiviral compounds against a virus because this requires inoculation of often highly infectious viruses into the cells and the production of additional infectious virus cells. Many viruses are extremely contagious and hazardous and therefore are difficult and sometimes impossible to screen compounds against as the intact virus is pathogenic and hazardous to humans, animals, or crops. Handling highly infectious material is not easy and not compatible with the high throughput process of screening large libraries of compounds which is necessary for cell-based screening of compounds. This severely limits the ability to screen a large number of compounds for broad-spectrum antiviral activity. An additional limitation to cell-based screening is that some viruses are difficult if not impossible to grow in a cell culture.

Therefore, there is a need for a method of discovering broad-spectrum antiviral compounds from large libraries of compounds. There is also a need for a method of developing a broad-spectrum antiviral drug, distributing the broad-spectrum antiviral drug to medical service providers, and treatment of viral infection in patients by medical service providers.

### SUMMARY

The present invention provides various aspects related to a method for development and delivery of a broad-spectrum antiviral drug and the treatment of a viral infection using the broad-spectrum antiviral drug.

In one aspect, the invention can be a method for identifying a broad-spectrum antiviral lead compound. The method includes determining antiviral activity of a plurality of compounds against two or more viruses and identifying a broad-spectrum antiviral lead compound from the plurality of compounds, said lead compound having activity against at least two of the two or more viruses.

In another aspect, the invention can be a method for identifying a class of broad-spectrum antiviral compounds. The method includes determining antiviral activity of compounds from two or more classes of compounds against two or more viruses. Each of the classes of compounds has one or more member compounds. The method also includes identifying a class of broad-spectrum antiviral compounds. The class of broad-spectrum antiviral compounds has a member compound with antiviral activity greater than a predetermined threshold activity level against a plurality of the two or more viruses.

In yet another aspect, the invention can be a method of rating compounds for broad-spectrum antiviral efficacy. The method includes determining antiviral activity for compounds against two or more viruses and rating each compound for broad-spectrum antiviral activity as a function of the determined antiviral activity and a number of viruses for which each compound has antiviral activity.

In another aspect, the invention can be a method for developing and marketing a broad-spectrum antiviral lead compound. The method includes selecting a broad-spectrum antiviral lead compound and developing a broad-spectrum antiviral drug from the broad-spectrum antiviral lead compound. The method also includes marketing the broad-spectrum antiviral drug to an aggregate of market opportunities. The aggregate of market opportunities includes addressing the treatment of viral infections associated with two or more viruses.

In another aspect, the invention can be a method for delivering a broad-spectrum antiviral compound to a drug company. The method includes identifying the broad-spectrum antiviral compound having antiviral activity against two or more viruses. The method also includes providing information to the drug company about broad-spectrum antiviral compound and an aggregate of market opportunities for the broad-spectrum antiviral compound. The method further includes providing a license to the drug company to produce and market a broad-spectrum antiviral drug from the broad-spectrum antiviral compound.

In yet another aspect, the invention can be a method for marketing a broad-spectrum antiviral drug to a health care provider. The method includes identifying the broad-spectrum antiviral drug having activity against two or more viruses and providing information about the broad-spectrum antiviral activity of the broad-spectrum antiviral drug to the health care provider. The method also includes delivering the broad-spectrum antiviral drug to the health care provider in response to receiving a request.

In still another aspect, the invention can be a method for treating a suspected viral infection in a patient by administering a broad-spectrum antiviral drug to a patient. The method includes determining a presence of the suspected viral infection associated with a virus in the patient. The method also includes administering the broad-spectrum antiviral drug to the patient wherein the administering of the broad-spectrum drug is prior to determining a particular virus responsible for the suspected viral infection.

In another aspect, the invention can be a method for treatment of a patient having a particular viral infection. The method includes determining an ineffectiveness of an available antiviral drug against a particular viruses associated with the particular viral infection and administering a broad-spectrum antiviral drug to the patient.

### BRIEF DESCRIPTION OF THE DRAWINGS

The present invention will become more fully understood from the detailed description and the accompanying drawings.

FIG. 1 is a flow chart illustrating one embodiment of a broad-spectrum compound screening and identification method.

FIG. 2 is a flow chart illustrating one embodiment of a broad-spectrum antiviral compound screening and lead broad-spectrum antiviral compound selection method.

FIG. 3 is a flow chart illustrating one embodiment of a method for developing and delivering a broad-spectrum antiviral drug for patient treatment.

FIG. 4 is a flow chart illustrating one embodiment of a method of treatment of a patient with a suspected viral infection.

FIG. 5 is a flow chart illustrating one embodiment of a method of treatment of a patient with a viral infection.

Corresponding reference characters indicate corresponding parts throughout the several views of the drawings.

### DESCRIPTION

Definitions

Following are exemplary definitions applicable to terms and acronyms within the detailed description. It should be understood that these definitions are only exemplary in nature and other definitions may also apply.

Amplicon: A circular DNA molecule that contains the viral origin of replication and is replicated within cells by borrowed replication factors required by the virus, e.g., trans-acting factors such as origin-binding proteins, replication enzymes, etc.

Broad Spectrum Antiviral (BSAV): A compound that inhibits the replication of or kills two or more separate and distinct viruses.

CC50: A standard of measure indicating the concentration of a compound that causes 50 percent of maximum cytotoxicity.

Cytotoxicity—Of, relating to, or producing a toxic effect on cells.

Defective genome: A DNA or RNA molecule that contains all the genetic elements (e.g., cis-acting elements) required for viral genomic replication and transcription, but lacks one or more of the genetic elements that encode the borrowed factors or enzymes (e.g., trans-acting factors) required for replication. Defective genomes require the addition of a missing factor in order to replicate.

Diploid cells—Cells that have one pair of each type of chromosome so that the basic chromosome number is doubled.

DNA: deoxyribonucleic acid

EC50: A standard measure of effective concentration (EC) which is the concentration of a compound required to achieve a 50 percent inhibition of replication of the virus, e.g., a reduction of 50 percent of the replication achieved in the absence of the compound. Sometimes used interchangeably with IC50.

IC50: A standard of measure of inhibitory concentration (IC) which is the concentration of a compound required to achieve a 50 percent inhibition of viral replication. IC50 is often used as interchangeable with EC50.

Inosine monophosphate dehydrogenase (IMPDH)

Minigenome: A type of defective or artificial genome that has an incomplete genome that contains all the genome sequence elements (e.g., cis-acting elements) that are required for replication of the viral RNA genome, but lack one or all of the coding regions of the viral genome.

Multiple subgenomic replication culture (MSRC): A cell culture that contains cells that contain two or more viral subgenomic replication systems.

Mixed viral replicon culture (MVRS): A cell culture that contains cells that contain two or more viral subgenomic replication systems.

Quantitative reverse transcription-polymerase chain reaction (qRT-PCR): A standard method of measuring the amount of a specific RNA molecule in a sample.

Quantitative Structure-Activity Relationship (QSAR): A method of quantifying the relationship between the chemical structure of a compound and its biological activity.

Replicon: A replicon is an RNA or DNA molecule that is derived from a viral genome that is capable of replicating within cells cultured in vitro. Replicons encode all the essential non-structural proteins contained within the virus itself, but lack one or more structural proteins or other functional elements required for full virus replication. Replicons encode non-structural proteins including all genome components (e.g., cis-acting components such as nucleotide sequences required for viral RNA replication, transcription, and translation) and all components (e.g., trans-acting viral components such as all of the enzymes and other proteins required for replication and transcription of the viral genome within a cell). As a replicon lacks one or more structural proteins or other functional elements, replicons are not infectious.

RNA: Ribonucleic acid

Reverse transcription polymerase chain reaction (RT-PCR)

Structure-Activity Relationship (SAR)

Selectivity Index (SI): A ratio of cytotoxic concentration (CC) over the effective concentration (EC). For example, SI=CC50/EC50.

Subgenomic Viral Replication System (SVRS): A model of the virus that is an incomplete viral genome capable of replication, which lacks one or more genetic elements that are essential for producing infectious virus particles.

Therapeutic Index (TI): Another name for the Selectivity Index (SI) which is a ratio of cell cytotoxic concentration (CC) over the effective concentration (EC).

Introduction

The present invention addresses a new and novel approach to the discovery of antiviral compounds having broad-spectrum antiviral efficacy, the development and marketing of broad-spectrum antiviral drugs, and the treatment of viral infections associated with a virus by a compound with broad-spectrum antiviral activity. Broad-spectrum antiviral compounds have antiviral activity or efficacy against two or more viruses. The identification of a compound having broad-spectrum antiviral activity is integral component to the methods described herein as the broad-spectrum methods describe a strategy, method, factors, criteria, market opportunities, regulatory approval for discovery and development of compounds demonstrating broad-spectrum antiviral activity and include the treatment of a virus infection of a patient by a medical care provider using the broad-spectrum antiviral compound.

A broad-spectrum antiviral compound is a compound with antiviral activity against two or more viruses from two or more distinct families of viruses. The viruses may be two or more viruses within the same order, family, subfamily, genus, subgenus or species, or may be from different viral orders, families, subfamilies, genus, subgenus or species. In one embodiment, the broad-spectrum antiviral may have antiviral activity against two or more viruses that may be two or more RNA viruses, two or more DNA viruses, or may be a combination of one or more RNA viruses and one or more DNA viruses. The combination may also include sub-combinations within the many DNA and RNA viruses. As one example, the viruses for which the broad-spectrum antiviral compound has antiviral activity may include antiviral activity against two or more positive-strand RNA viruses, two or more negative-strand RNA viruses, or a combination of one or more positive-strand RNA viruses and one or more negative-strand RNA viruses. Generally, broad-spectrum is intended to include any combination or grouping of two or more viruses, wherein at least two of the viruses are distinct from one another.

As will be discussed further, the method of broad-spectrum antiviral compound screening is a cell-based screening that provides a novel method of screening by assays, a very large number of potential antiviral compounds against multiple viruses. The cell-based assay method provides quantitative data and measurements of antiviral activity for each and every compound, each and every compound dilution, against each and every virus in each and every host cell of the screening process. As such, broad-spectrum antiviral screening method provides a significant amount of data which heretofore has not been developed and made available for the identification of antiviral compounds from a very broad compound library.

The broad-spectrum antiviral method is also benefited by the novel utilization of one or more Subgenomic Viral Replication Systems (SVRS) which are representative of one or more viruses. A Subgenomic Viral Replication System (SVRS) is an incomplete viral genome capable of replication, which lacks one or more genetic elements that are essential for producing infectious virus particles. By screening a Subgenomic Viral Replication System (SVRS) representative of a virus, the broad-spectrum antiviral method enables the screening process to include screening of numerous compounds for antiviral efficacy against two or viruses many of which heretofore have not been easily or effectively assayed. By utilizing one or more Subgenomic Viral Replication Systems (SVRS) in lieu of the live virus, assays may be performed against viruses which are dangerous and highly hazardous. Additionally, performing antiviral assays against a Subgenomic Viral Replication System (SVRS) provides the opportunity to utilize high-throughput and often highly automated assay methods. As a result, assays may be performed against a significantly greater number of potential antiviral compounds at varying dilution levels, against a greater number viruses and host cells at significantly lower costs and in shorter time periods. Additionally, such methods also provide improved quality control and effectiveness of the assays, and more accurate and precise data. It also allows the effectiveness of two or more antiviral compounds to be compared.

The identification of one or more broad-spectrum antiviral compounds by the broad-spectrum antiviral screening provides the new opportunities to develop new drugs that may be administered for the treatment of viral infections in patients. In the past, an antiviral compound has been identified which has antiviral activity against a particular virus. Drug companies test one or more variations of the compound in an effort to develop a drug that provides effective and safe antiviral medication against a particular and known viral infection in a patient. However, many viruses are not common and do not warrant the attention and cost required to develop and bring to market an antiviral drug from a compound demonstrating antiviral activity against one particular virus. The broad-spectrum antiviral method significantly improves this compound to drug development and approval process. By identifying compounds that have antiviral activity against two or more viruses, two or more drug market opportunities may be aggregated which allow for the sharing of cost.

Additionally, the development and availability of a drug with broad-spectrum antiviral activity provides for improvements in the treatment of virus infections. This includes enhancing the antiviral effectiveness of virus-specific drugs in a cocktail treatment. Additionally, a broad-spectrum antiviral drug provides the ability to treat unknown or undiagnosed viral infections in patients. Such a treatment by a broad-spectrum drug provides medical service providers and their patients with the opportunity for treating a viral infection at a very early stage which may significantly inhibit the replication of the virus and therefore the negative effects or damage of the virus including reducing the spread of the virus.

The broad-spectrum antiviral screening and discovery method includes selection of the screening method, selection of two or viruses to be screened, selection of one or more Subgenomic Viral Replication Systems (SVRS) representative of the two or more viruses, selection of the appropriate host cells, selection of the compounds to be screened for broad-spectrum antiviral efficacy, selection of the assay method, selection of the screening method, collection of quantitative data indicative of broad-spectrum antiviral activity, analysis of the data including rating and ranking compounds and classes of compounds, and identification of lead compounds and/or classes of compounds for broad-spectrum antiviral drug development. Each of these will now be addressed in greater detail.

Broad-Spectrum Screening

The broad-spectrum antiviral compound discovery and screening method is a cell-based unbiased approach to compound assaying and screening. Compounds are assayed against host cell and viruses for identification of compounds that inhibit viral replication without concern or knowledge of a particular target. The cell-based approach utilizes screening of virus cultures, which are intracellular pathogens, as viruses only replicate within a host cell. Cell-based screening in the broad-spectrum antiviral screening method is different from traditional antiviral compound screening methods which have generally been target-based screening. In the target-based approach, a particular biochemical target is selected which is believed to be associated with one or more characteristics of the virus such as replication. Candidate antiviral compounds are screened for inhibition of the particular target, thereby implying an inhibition of replication. The target is often an enzyme or a receptor that is known or thought to be essential to the process of viral replication. However, the selected target may or may not be directly or indirectly associated with viral replication.

Although cell-based screening has been used successfully throughout the drug-discovery field, it has generally been believed to be difficult if not impossible to utilize in the screening of antiviral compounds against many of the most desired viruses for which antiviral compounds are sought. This is because cell-based screening of a virus requires inoculation of an infectious virus onto host cells and the replication and production of additional infectious progeny virus. The handling of such infectious material is not easy and is not compatible with the high throughput assay or screening process against large numbers of compounds which is necessary for cell-based screening. The broad-spectrum antiviral compound discovery and screening method provides for the ability to utilize cell-based screening of large numbers of compounds against multiple viruses, in part due to the use of Subgenomic Viral Replication Systems (SVRS) in lieu of one or more of the viruses in the screening process.

In one embodiment, the broad-spectrum antiviral method utilizes cell-based assays that may include one or more Subgenomic Viral Replication Systems (SVRS) in the primary assay, and then utilizes biochemical assays as the secondary screening method to narrow the selection process or to verify broad-spectrum antiviral activity. This is different from screening using biochemical assays as the primary assays and cell-based assays and mechanisms of action in the secondary assays.

In one embodiment, the broad-spectrum antiviral method provides for the identification of a broad-spectrum antiviral lead compound by determining antiviral activity for each of a plurality of compounds against each of two or more viruses or Subgenomic Viral Replication Systems (SVRS) representing one or more of the two or more viruses. The identified antiviral efficacy or activity is preferably in the form of quantifiable antiviral activity data measured through chemical assays of each compound on each virus and one or more host cells. For example, such determined antiviral activity may be a quantified measurement of antiviral activity such indicated by standard measurements such as an effective concentration, a cell cytotoxic concentration, and a Selectivity Index (SI).

The identified antiviral efficacy may also, in the alternative, be determined or obtained from another source or from an entity responsible for assaying chemicals against one or more viruses. However, for consistency and effectiveness, it is desirable for the determined antiviral activity to be based on a standardized assay platform or process such that all or most determinations are based on the same or a common set of factors and procedures.

A broad-spectrum antiviral lead compound is identified from the plurality of compounds based on the determined antiviral efficacy of each of the plurality of compounds against each of the two or more viruses. The identified broad-spectrum lead compound is one that has antiviral activity against at least two of the two or more viruses which may include tested antiviral activity against one or more Subgenomic Viral Replication Systems (SVRS) which are representative of at least one or more of the two or more viruses. An identified broad-spectrum antiviral lead compound may have antiviral activity greater than a predetermined threshold antiviral activity against two or more of the viruses.

In another embodiment, a broad-spectrum antiviral compound has demonstrated antiviral efficacy against two or more viruses from a single viral family, genus, or subgenus. In such a case, the identified broad-spectrum antiviral lead compound may also have antiviral activity greater than a predetermined threshold antiviral activity against at least two viruses of the two or more viruses from the single viral family, genus or subgenus.

In broad-spectrum antiviral screening, a broad-spectrum antiviral lead compound may be identified by a number of different methods including a function of a rating of each compound for broad-spectrum antiviral activity. In such a rating system, as will be addressed in further detail below, the “broad-spectrum” rating considers not just the antiviral efficacy against a single virus but rates each compound for efficacy across a plurality of viruses. For example, a broad-spectrum antiviral rating of a compound or class of compounds will be a function of the number of viruses for which the each compound has antiviral activity. In such a case, the identification of a broad-spectrum antiviral lead compound may be the identification and/or selection of a compound that has antiviral activity greater than a predetermined threshold antiviral activity against each of the at least two of the two or more viruses or subgenomic viral replications system representing one or more of the at least two of the two or more viruses.

In another embodiment, the broad-spectrum screening method identifies a compound or a class of compounds that have broad-spectrum antiviral efficacy. The method includes the determination of antiviral activity of each compound against two or more viruses which may include one or more subgenomic viral replication systems representative of one or more of the two or more viruses. Such a compound may be from two or more classes of compounds where each class of compounds would typically have one or more member compounds. The method also includes the identification of a class of broad-spectrum antiviral compounds that has a member compound with antiviral activity greater than a predetermined threshold level against two or more of the two or more viruses.

The identification of a class of broad-spectrum antiviral compounds may be a function of identifying compounds within a class that demonstrate antiviral activity against two or more viruses from a single viral family. In such an embodiment, the identified class of broad-spectrum antiviral compounds may also have antiviral activity greater than a predetermined threshold antiviral activity against two or more viruses of the two or more viruses from the single viral family.

In another embodiment, broad-spectrum antiviral screening method includes the selection of a member virus from two or more virus families to act as an initial model for each virus family during screening. Once the selected family members are determined, one or more Subgenomic Viral Replication Systems (SVRS) are selected and/or produced to represent one or more of the selected family members. Each Subgenomic Viral Replication Systems (SVRS) is selected and produced as a function of optimizing broad-spectrum antiviral detection, analysis and identification. The method also includes screening a library of compound classes against the selected virus family members. The screening may be performed, in one embodiment, in a multi-stage screening process designed to identify compound classes with at least one active member against two or more Subgenomic Viral Replication Systems (SVRS) representing two or more differentiable viruses.

In another embodiment, the broad-spectrum antiviral method includes determining one or more relationships between individual viruses rather than groups of viruses thereby providing for development of drugs that have antiviral activity against two or more viruses. This is different from simply screening compounds against two viruses.

As will be discussed in more depth below, the processes, decisions and factors used during this process are unique to the broad-spectrum antiviral process as all considerations relate to identifying compounds that have broad-spectrum antiviral efficacy. If a compound is assayed and it demonstrates high anti-viral activity against a single virus, but not others, the compound is excluded from further broad-spectrum screening. High efficacy against a single virus may be desirable for a target-based screening program and a virus-based efficacy, but it is not indicative of broad-spectrum antiviral activity.

Selection of Viruses for Broad-Spectrum Screening

Selection of the two or more viruses to be used in the broad-spectrum antiviral screening method is a function of one or more broad-spectrum factors and may, for example, be based on an established decision process, such as a quantitative weighted selection process. Broad-spectrum virus selection factors may include in one embodiment viruses from both positive and negative-strand RNA viruses. In such a case, the desired broad-spectrum antiviral compound would be one that is active against all forms of RNA viruses. In another embodiment, the viruses to be screened in the broad-spectrum antiviral screening method include the global list of known and unknown viruses in humans including DNA and RNA viruses. However, the methods described herein are not limited to human viruses and as such animal, insect, and plant viruses would also be considered for the broad-spectrum antiviral compound screening and drug development as described herein. As such, the method herein is applicable to any and all viruses that cause any disease, whether in man, animal, plant, or otherwise.

Representative viruses for broad-spectrum compound screening within these families are selected as a function of factors or considerations. For example, screening of a virus that is well studied with basic research would be a virus that would be suitable for broad-spectrum compound screening and drug development. Additionally, another factor would be the classification of hazardous of a virus. For example, a virus having a lower level biohazard classification may be preferable over a virus having a higher level biohazard classification. Typically lower level biohazard classification provides for fewer restrictions on handling and therefore provides for improved and less costly antiviral screening. Similarly, another factor to consider is the ease of manipulation of the virus. Another factor to consider in the selection of a virus for broad-spectrum antiviral screening is the ability of the virus to grow or replicate in a cell culture. Some viruses replicate readily in a cell culture which provides the ability to assay the virus in a cell culture assay to determine antiviral efficacy of a compound using high-throughput and highly mechanized system. However, other viruses do not replicate well in a cell culture and in fact some viruses will not replicate at all in a cell culture, but will only replicate in a living tissue. These viruses may not be as desirable for broad-spectrum assaying as others.

Another factor considered is the availability of a good and reliable viral assay for the virus. While viral assays may be readily available for some viruses, others are difficult to obtain and are not readily available for broad-spectrum screening. Yet another factor considered in the selection of a virus to include in broad-spectrum screening is the availability or ability to produce a subgenomic viral replication system (minigenome, replicon or otherwise). In lieu of live virus cultures themselves, as will be discussed later, Subgenomic Viral Replication Systems (SVRS) may be used in the broad-spectrum screening method. These and other factors to be considered in the selection of the two or more viruses and any Subgenomic Viral Replication Systems (SVRS) for the broad-spectrum antiviral screening method include:
a. reliability of the virus or Subgenomic Viral Replication System (SVRS),
b. signal-to-noise ratio of the indicator indicating viral replication,
c. reliability of the dose-response with positive control (e.g., a known inhibitor) on a well-to-well, plate-to-plate, and experiment-to-experiment basis,
d. ease of manipulation of the virus or Subgenomic Viral Replication Systems (SVRS),
e. amenability to automation or automated laboratory screening,
f. availability of indicator cells which may be prepared by transient transfection of cells with the Subgenomic Viral Replication Systems (SVRS) or a stable cell line is prepared that contains the minigenome/replicon,
g. ability to freeze batches of cells in advance,
h. reliable quality from batch-to-batch,
i. stability or a signal strength from passage to passage, and
j. ability to attach to tissue culture plates.

These virus selection criteria apply to the selection of any virus including positive-strand RNA viruses, negative-strand RNA viruses, retroviruses, DNA viruses, viroids, and other types of viruses that are desired to be screened for broad-spectrum antiviral activity.

A Subgenomic Viral Replication System (SVRS) is a model of the virus that is an incomplete viral genome capable of replication, which lacks one or more genetic elements that are essential for producing infectious virus particles. A Subgenomic Viral Replication System (SVRS) is non-infectious and may be used in place of the live virus for broad-spectrum antiviral screening. Subgenomic Viral Replication Systems (SVRS) include replicons, defective genomes, and minigenomes, but also include future Subgenomic Viral Replication Systems (SVRS) which are capable of modeling the replication process of viruses with containing the virus itself. As will be discussed later, replicons are capable of self-replication whereas defective genomes including minigenomes require the addition of a missing factor in order to replicate. While Subgenomic Viral Replication Systems (SVRS) are known, prior Subgenomic Viral Replication System (SVRS) development focused on a few select viruses and has not addressed the screening of compounds for broad-spectrum antiviral activity.

As noted, one form of a non-infectious Subgenomic Viral Replication System (SVRS) is a defective genome. Defective viral genomes are cells that contain all the genome elements (e.g., cis-acting elements) required for viral genomic replication and transcription, but lack one or more of the genetic elements that encode the borrowed factors (e.g., trans-acting factors) required for replication. Such defective genomes, therefore cannot replicate by themselves, e.g., they are not replicons, but defective genome cells are replicated if the missing factor or factors are supplied “in trans.” A cell that contains the defective genome plus the necessary trans-acting factors exhibits a functional similarity to a replicon in that partial viral replication occurs within the cell and no infectious virus is produced. As with cell cultures containing replicating replicons, cell cultures containing replicating defective viral genomes represent a useful tool for antiviral drug discovery. Examples of defective genomes include the genomes contained within defective interfering virus particles that have been observed for many RNA and DNA viruses such as Alpha viruses (e.g., Sindbis virus) and herpes viruses (e.g., herpes simplex virus type one).

Another example of a defective genome is a minigenome. A minigenome is a type of artificial genome that has an incomplete genome that contains all the genome sequence elements (e.g., cis-acting) that are required for replication of the viral RNA genome, but lack one or all of the coding regions of the viral genome. Minigenomes have been constructed for negative-strand RNA viruses including respiratory syncytial virus (RSV), rabies virus, and measles virus.

Another example of defective viral genomes is an “amplicon” of a DNA virus. Amplicons are circular DNA molecules that contain the viral origin of replication and are replicated within cells by the borrowed replication factors (e.g., transacting factors such as origin-binding proteins, replication enzymes, etc.) required by the virus. Amplicons have been developed for herpes viruses (HSV-1) and other DNA viruses such as Papova viruses (e.g. simian virus 40 (SV40).

Another Subgenomic Viral Replication System (SVRS) is a viral “replicon.” A replicon is derived from a viral genome that is capable of replicating within cells cultured in vitro. Replicons encode all non-structural proteins contained within the virus itself. These include all genome elements (e.g., cis-acting elements such as nucleotide sequences required for viral RNA replication, transcription, and translation) and all of the trans-acting components (e.g., trans-acting viral components such as all of the enzymes and other proteins required for replication and transcription of the viral genome within a cell). However, replicons lack one or more functional factors required for full virus replication, and therefore replicons do not replicate and are not infectious. The factor that is missing may be absent due to a deletion of all or part of the sequence encoding that function, or the factor may be functionally missing due to a mutation, such as a point mutation, thereby rendering the factor nonfunctional.

Replicons capable of persistent replication in cells may be created that contain persistently replicating viral replicons for many viruses. Examples of one subset of viruses and available viral replicons are illustrated in Table 1.

TABLE I
Viral Replicons for Broad-spectrum Antiviral Screening

| | | Infec- | Non- |
|---|---|---|---|
| | | tious | cytopathic |
| Family | Virus (Common) | clone | Replicon |
| Togaviridae | Sindbis | yes | yes |
| | Rubella | yes | possible |
| | VEEV | yes | possible |
| | WEEV | | possible |
| | EEEV | | possible |
| | Marayo O'nong nong | | |
| | Ross River | | |
| | Chikungunya | | |
| Picornaviridae | Poliovirus | yes | yes |
| | Coxsackirus | yes | possible |
| | Enterovirus | yes | possible |
| | Hepatitis A | yes | possible |
| | Rhinovirus | | |
| Flaviviridae | Yellow fever | yes | yes |
| | Dengue fever | yes | yes |
| | West Nile | yes | yes |
| | Japanese Encephalitis | yes | yes |
| | Hepatitis C | yes | yes |
| | Tick-born encephalitis | | |
| | Murray Valley | | |
| | Omsk HF | | |
| | Kyasanur forest | | |
| | SLE | | |
| Astroviridae | Astrovirus | yes | possible |
| Rhabdoviridae | Rabies | yes | possible |
| Orthomyxoviridae | Influenza A | yes | yes |
| | Influenza B | | possible |
| Paramyxoviridae | Respiratory syncytial (RSV) | yes | yes |
| | Measles | yes | possible |
| | Mumps | yes | possible |
| | HPIV | | possible |
| | HMPV | | possible |
| | Nipah | | |
| | Hendra | | |
| | Metapneumovirus | | |
| Filoviridae | Ebola | yes | yes |
| | Marburg (MBGV) | | possible |
| Bunyaviridae | La Crosse | | possible |
| | California encephalitis | yes | possible |
| | Hantaan | | possible |
| | Crimean-Congo | | possible |
| | Rift Valley fever | | possible |
| | Sin nombre | | |
| Arenaviridae | Lassa fever | | possible |
| | Argentine Hemorrhagic fever | | possible |
| | Bolivian Hemorrhagic fever | | possible |
| | Junin | | |
| | LCMV | | possible |
| Bornaviridae | borna disease virus | yes | possible |
| Reoviridae | Colorado tick fever | possible | possible |
| Hepadnaviridae | Hepatitis B | yes | yes |
| Papillomaviridae | Human papilloma | yes | yes |
| Polyomaviridae | JC | yes | possible |
| | BK | yes | possible |
| Herpeviridae | Herpes simplex type one | yes | yes |
| | (HSV-1) | | |
| | Herpes simplex type two | yes | possible |
| | (HSV-2) | | |
| | Epstein-Barr (EBV) | yes | yes |
| | Human cytomegalovirus | yes | possible |
| | (HCMV) | | |
| | Varicella-zoster (VZV) | yes | possible |
| | Human herpes type six (HHV6) | possible | possible |
| | Human herpes type seven | possible | possible |
| | (HHV7) | | |
| | Human herpes type eight | possible | possible |
| | (HHV8) | | |
| Adenoviridae | Human adenovirus | yes | possible |
| Retroviridae | Human immunodeficiency | yes | possible |
| | type one | | |
| | (HIV-1) | | |
| | HIV type two (HIV-2) | yes | possible |
| | Human t-cell leukemia type one | yes | possible |
| | (HTLV-1) | | |
| | Human t-cell leukemia type two | yes | possible |
| | (HTLV-2) | | |
| Parvoviridae | Human parvovirus | yes | possible |
| | Adeno-associated virus | yes | yes |
| Caliciviridae | Feline | | |
| | Murine | | possible |
| | Norwalk | | |
| | HEV | | |
| Coronaviridae | Bovine | | possible |
| | Murine | yes | possible |
| | Human coronavirus | | possible |
| | SARS | yes | possible |

As indicated, a Subgenomic Viral Replication System (SVRS) may be representative of any virus that infects the cells of any organism from any eukaryotic kingdom, phyla, or family, including plants, fungi, insects, and protists. In another embodiment, it is envisioned that the broad-spectrum method will be adaptable for any virus that cannot now be made into a Subgenomic Viral Replication System (SVRS) but could be made into such a system in the future which includes human, animal and plant viruses. Other virus families and viruses for which the broad-spectrum methods apply include those known DNA and RNA viruses and families such as those identified at: http://www.ncbi.nlm.nih.gov/Taxonomy/Browser/wwwtax.cgi?name=Viruses. Additionally, the broad-spectrum is also applicable to a virus that may not be currently known.

If a Subgenomic Viral Replication Systems (SVRS) is not readily available for the desired virus, one or more Subgenomic Viral Replication Systems (SVRS) may, in some cases be developed. When one Subgenomic Viral Replication Systems (SVRS) is not available to represent a particular virus, a Subgenomic Viral Replication Systems (SVRS) may be developed as will be discussed below. Such development may utilize similar methods or techniques that have been developed for other Subgenomic Viral Replication Systems (SVRS). However, in other cases, the creation of a Subgenomic Viral Replication Systems (SVRS) is more difficult and therefore this should be considered in the selection of the viruses to be included in the broad-spectrum screening process.

Once the two or more viruses have been selected inclusion in the broad-spectrum method, one or more Subgenomic Viral Replication Systems (SVRS) may be selected for the cell-based broad-spectrum screening. If only one Subgenomic Viral Replication System (SVRS) system is available for a particular virus, screening may be performed with the one that is first available. If both a minigenome and a replicon are available for a particular virus, the Subgenomic Viral Replication Systems (SVRS) should be selected that demonstrate a higher level of reliability is assays and that is easier to handle during the assay and screening stages. In general, broad-spectrum antiviral screening is performed with the Subgenomic Viral Replication System (SVRS) that more clearly and accurately represent the virus with regard to replication and therefore antiviral efficacy. If both Subgenomic Viral Replication Systems (SVRS) perform equally well, a replicon system is preferred, because replicon systems contain a higher number of viral proteins, which therefore provides for a higher number of targets for consideration of antiviral efficacy of each compound. However, for one or more viruses, minigenome systems are considered easier to develop than a similar replicon. Another factor is the ability to confirm the activity of the viral assay. Additionally, for certain types or families of viruses, one Subgenomic Viral Replication System (SVRS) may be desirable over another. For example, minigenomes may be the only systems available at a particular time a negative-strand virus. As such, minigenomes may be the preferred Subgenomic Viral Replication System (SVRS) if negative-strand viruses are to be included in the broad-spectrum screening method. However, if replicon systems later become available, this selection should be reevaluated.

In order to evaluate broad-spectrum antiviral efficacy, each virus and Subgenomic Viral Replication System (SVRS) used in the broad-spectrum antiviral method should generally be genetically distinct from any other being evaluated. A virus or Subgenomic Viral Replication System (SVRS) may be in different virus order, family, subfamily, genus, subgenus or species, or may be in the same order, family, subfamily, genus, subgenus or species. They may be the same virus but having a different genotype, or may be a mutant of same virus. In the case of Subgenomic Viral Replication Systems (SVRS), each may differ by as little as one base pair.

The availability of one or more Subgenomic Viral Replication Systems (SVRS) enables the broad-spectrum method to include the assaying of antiviral compounds against highly hazardous and infectious viruses in an infection-free manner. Utilizing a Subgenomic Viral Replication Systems (SVRS) in lieu of a native virus, the particular virus represented by the Subgenomic Viral Replication Systems (SVRS) may be included in a high-throughput screening against a greater number of possible compounds. Also utilizing one or more Subgenomic Viral Replication Systems (SVRS) to screen candidate antiviral compounds against multiple viruses provides an opportunity to screen a significantly greater number of candidate compounds against a greater number of virus using high-throughput screening methods. The broad-spectrum method produces quantifiable data that enables the comparison of the effects of a candidate antiviral compound on each tested virus to determine the antiviral activity against a broad-spectrum of or multiple viruses of multiple families of viruses. Thus, the broad-spectrum antiviral method provides quantified data and information on the specificity of the antiviral effect for each candidate antiviral compound of a compound library against two or more viruses. This information is helpful, for example, in assessing whether the candidate antiviral compound is inhibiting a specific viral target associated with viral replication or on a cellular target and thus exerting its effect on the viruses indirectly.

As discussed above, in many cases, a suitable Subgenomic Viral Replication System (SVRS) may not be readily available. As such, the broad-spectrum antiviral method includes consideration for the development of Subgenomic Viral Replication Systems (SVRS) which may be available for broad-spectrum screening.

The broad-spectrum antiviral method that includes the use of Subgenomic Viral Replication Systems (SVRS) enables the inclusion of viruses in the screening process that are highly hazardous including viruses that are rated at biohazard levels of BL3 and BL4. As such, the broad-spectrum antiviral method enables the screening process to include a significantly expanded library of compounds. As such, broad-spectrum antiviral method uniquely address biolevel safety limitations associated with BL3 and BL4 viruses. This significantly speeds up more extensive testing of compounds against viruses that are difficult if not impossible to assay, e.g., BL3 and BL4 classified viruses.

Additionally, some viruses or replicons of the virus cannot be replicated in a laboratory environment. For example, the hepatitis C virus (HCV) only replicates in a living human or chimpanzee body. In such a case, the broad-spectrum screening method of one or more embodiment of the present invention provide for screening or assaying replicons that are within the families of these viruses that can not be replicated in the laboratory.

The broad-spectrum method further provides for reducing the number of compounds required for assaying BL 3 and BL 4 viruses by focusing the BL3 and BL4 assays on the compounds that have first passed the high throughput assaying for BL2 representative viruses which resulted in broad-spectrum antiviral efficacy against two or more representative BL2 viruses. The broad-spectrum antiviral method also solves the problem of finding an antiviral compound or agent against a virus that cannot be replicated easily in the laboratory such as BL3 and BL4 viruses.

Cell cultures comprising Subgenomic Viral Replication Systems (SVRS) offer a number of benefits in the discovery and analysis of antiviral compounds. They permit the effect of an antiviral compound to be observed in the context of living cells, so that any compounds that show antiviral activity necessarily enter and act within living cells. Subgenomic Viral Replication System (SVRS) cell cultures also allow the immediate identification of antiviral compounds with obvious undesirable cytotoxicity using well established cytotoxicity assays. These cell cultures also permit cell-based drug discovery screens and other studies to be performed against viruses such as hepatitis C virus (HCV) and human papillomavirus (HPV) that are unable to be conventionally cultured in vitro. Because viral functions related to infectivity are typically not required for viral genome replication, viral replicons lacking at least one infectivity-related sequence are much safer and thus easier to work with than infectious virus. An infectivity-related sequence is a sequence that encodes a protein required for the virus to infect a cell.

Another advantage of the Subgenomic Viral Replication System (SVRS) cell cultures is that the Subgenomic Viral Replication Systems (SVRS) may be genetically manipulated to comprise heterologous sequences such as those encoding reporter genes such as luciferase, beta-galactosidase, secreted alkaline phosphatase, betalactamase, or green fluorescent protein that facilitate high throughput automated analysis of viral genome replication.

In practice, it may also be desirable to provide a control cell culture along with a multiplexed assay which does not contain the one or more Subgenomic Viral Replication Systems (SVRS). In this manner, it is possible to measure the effect of the candidate antiviral compound on the host cells themselves.

Replicon Development

For the broad-spectrum antiviral method, the selection and/or production of a Subgenomic Viral Replication Systems (SVRS) includes the selection of a marker gene, resistance gene, or reporter gene that may be replicated along with the Subgenomic Viral Replication Systems (SVRS). The selection of markers for Subgenomic Viral Replication Systems (SVRS) for the broad-spectrum antiviral method is a function of factors related to determining broad-spectrum antiviral efficacy of compound. This is different from target-based screening wherein once a replicon is identified or developed for a single target or virus, the replicon selection and development process stops. With the broad-spectrum antiviral method, a marker is selected such as to enable the production of more than one replicon such that a common marker is used for multiple replicons. As such, one embodiment of the broad-spectrum antiviral replicon selection and development method includes selection of markers based on a function and factors associated with multiple replicons as an output, not just a single replicon. For broad-spectrum antiviral a replicon is not simply selected for a virus where it is determined that a suitable replicon is representative of the virus, and a single suitable replicon is not used for the assay and testing.

The selection of the marker gene for a Subgenomic Viral Replication System (SVRS) is also based on criteria that optimize the portfolio of Subgenomic Viral Replication Systems (SVRS) utilized in the assays to identify broad-spectrum activity. Broad-spectrum antiviral screening is not simply looking at the efficacy against a single virus. Even though a Subgenomic Viral Replication System (SVRS) for one virus is identified, that replicon is not automatically used for assaying. Further replicon development is required for broad-spectrum antiviral cell-based screening. While one suitable replicon for a single virus may be identified, that one replicon may not be desirable as it may not be applicable to other viruses for development of other replicons. As such, the selection process for the reporter genes and replicons continue until a suitable replicon and reporter gene is identified that will be applicable to two or more viruses and therefore two or more replicons. As such, with the broad-spectrum antiviral method, it is not necessary to determine if there is a better reporter gene or replicon. In the broad-spectrum antiviral method the focus is identifying and selecting a process or marker gene that may be common to two or more viruses. By identifying one or more reporter genes that may be utilized to develop multiple replicons associated with multiple viruses, broad-spectrum antiviral screening provides improved effectiveness and efficiency associated with the screening of the many compounds against the multiple viruses.

In selection of markers for Subgenomic Viral Replication Systems (SVRS) in broad-spectrum screening, a single marker may also not satisfy broad-spectrum antiviral screening requirements. The process of identifying a single reporter gene and replicon production method is complicated because a reporter gene that may be suitable for one virus may not be suitable for a second or third virus. For broad-spectrum antiviral screening, the selection is based on identification of a marker that has the cumulative best properties to provide a common marker applicable to all viruses to be assayed, rather than the selection of one that is best for a particular replicon or virus or assay.

Reporter genes are typically nucleic sequences encoding easily assayed proteins. They are used to replace other coding regions whose protein products are difficult to assay. Reporter genes may be attached to other sequences so that only the reporter protein is made or so that the reporter protein is fused to another protein, e.g., a fusion protein. Reporter genes report a variety of different properties and events including: the strength of promoters, whether native or modified for reverse genetic studies, the efficiency of gene delivery systems, the intracellular fate of a gene product, the interaction of two proteins in the two-hybrid system or of a protein and a nucleic acid in the one-hybrid system, the efficiency of translation initiation signals, and success of molecular cloning efforts.

In one embodiment of the present invention for a broad-spectrum antiviral screening method, a marker is selected based on factors and criteria to provide for the selection of a marker that is common to multiple viruses or Subgenomic Viral Replication Systems (SVRS). In order to select a marker that is applicable to the desired set of applicable replicons, the process, factor, criteria, strategy and decision process for selecting the marker is different. For broad-spectrum antiviral, the selection includes:

A selection marker gene may be any gene which provides the identification of a difference in cells for selection of the cells indicative of a particular characteristic. Selection marker genes include a resistant gene and a reporter gene, both of which are useful in the development of Subgenomic Viral Replication Systems (SVRS) in screening for broad-spectrum antiviral activity.

A resistance gene provides an output or product which gives a cell the ability to resist a certain drug or compound. Resistance genes include genes encoding dihydrofolate reductase, puromycin acetyl transferase, neomycin phosphotransferase, blastocidin S, hygromycin B phosphotransferase, guanine phosphoribosyl transferase, and zeocin resistance protein. Expression of these genes confers resistance to methotrexate puromycin, G418, blastocidin, hygromycin B, mycophenolic acid, and zeocin, respectively.

The cells without the resistance gene cannot survive in the presence of the drug. The resistance gene may be cloned into a replicon or other Subgenomic Viral Replication Systems (SVRS). A replicating replicon may produce the gene product including the resistance gene thereby making the host cell resistant to the drug. Cells that do not contain the replicated replicon with the resistance gene are killed by the drug, resulting in only cells containing the replicated replicon.

Resistance genes are useful in constructing and assaying a Subgenomic Viral Replication System (SVRS) as the resistance gene provides the ability to select out the cell containing replicated replicons from cells not containing a replicon. This is important because, while Subgenomic Viral Replication Systems (SVRS) replicate in host cells, their replication rate is not high. For example, when 2.5 million celia are transfected with West Nile virus replicons, only 3 colonies grow that contain the West Nile replicon. Each colony representing replication of a single cluster of cells grows from a single replicon cell. As such, it is desirable for the replicon to include a resistance gene that enables the selection of the replicated Subgenomic Viral Replication Systems (SVRS) for Subgenomic Viral Replication System (SVRS) development as well as assay assessment. A resistance gene may also be used for reporting the quantification of replication, but usually genes denoted as reporter genes provide a stronger signal. For example, the Neo gene product, NPT2 protein, may be quantified by using the resistance gene NPT2 ELISA.

A reporter gene product is a gene that provides a strong signal indicating presence of the reporter gene and therefore the Subgenomic Viral Replication System (SVRS) that includes the reporter gene. A strong signal of the reporter gene improves the assay process and the accuracy of the results of the assay. Examples of reporter genes include Renilla luciferase, Firefly luciferase, alkaline phosphatase, β-glatosidase, GFP (Green fluorescence Protein). A Subgenomic Viral Replication System (SVRS) containing a reporter gene provides the ability to evaluate or determine the presence of the Subgenomic Viral Replication System (SVRS) or cell containing the reporter gene and therefore provides the ability to quantify the amount of Subgenomic Viral Replication System (SVRS) replication. Reporter genes may also be used in some situations, for cell selection. For example, GFP emits a green light in the presence of a UV light. As such, a UV light may be used in conjunction with GFP to separate the GFP positive cells from negative cells using a flow cytometer.

Several fusion proteins that include both a resistant gene and a reporter gene have been developed that demonstrate both resistance and reporter characteristics. These include hRUPac, which is a fusion gene containing humanized renilla luciferase (a reporter gene), ubiqutin-derived peptide (a proteolytic cleavage site), and pac (a resistance gene). In these as well as other embodiments, both reporter and resistant activities have been detected.

In other cases, determining a reporter or resistant gene is difficult because the constructed Subgenomic Viral Replication System (SVRS) may not replicate in some host cells. Therefore, it is necessary to test each Subgenomic Viral Replication System (SVRS) containing a resistant gene and/or a reporter gene to ensure replication. Based on the information obtained from replicating the Subgenomic Viral Replication System (SVRS), each resistance gene and reporter gene may be evaluated for the ability to replicate and then prioritized as to the desirability and opportunity for application and use in one or more Subgenomic Viral Replication Systems (SVRS).

The development of multiple Subgenomic Viral Replication Systems (SVRS) may also utilize a single Subgenomic Viral Replication System (SVRS) for a single virus such as Yellow Fever to evaluate and/or develop one or more resistance genes, reporter genes, and their combination for the development of other Subgenomic Viral Replication Systems (SVRS). For example, using the broad-spectrum antiviral method, a correlation among the HCV, Yellow Fever, West Nile, and Dengue viruses has been demonstrated. As such, a resistance-reporter fusion gene that was identified for a Subgenomic Viral Replication Systems (SVRS) for the Yellow Fever virus may be used for the development of one or more Subgenomic Viral Replication Systems (SVRS) for HCV, West Nile and/or Dengue and potentially other viruses. However, such a solution may not be applicable to other cases where the same resistance gene or reporter gene may not produce the same effects.

The selection and combinations of resistance genes and reporter genes may also be dependent on the assay or screening method selected for broad-spectrum antiviral assessment. For example, if an assay method utilizes a single virus in a single well for screening, the replicon selected may be common to any number of viruses. In practice, the resistance gene and/reporter gene and therefore replicon or Subgenomic Viral Replication Systems (SVRS) to be assayed will likely be selected based on ease of use, strength of signal, availability, or cost. However, when an assay is to utilize multiple viruses or Subgenomic Viral Replication Systems (SVRS) within a single assay well, e.g., a multiplexed assay, a different reporter gene for each virus or Subgenomic Viral Replication Systems (SVRS) may be required in order to provide flexibility in the assay process and to ensure that accurate and effective quantification for each viral replication within the multiplexed assay. In one or more embodiments, broad-spectrum antiviral screening replicons are selected or produced in a manner that is applicable to more than one replicon and/or more than one virus. The replicon production process and method determines which cells to use such that multiple replicons are possible. These steps and processes, replicon selection strategy and concepts are focused on broad-spectrum screening and drug development.

Host Cell Selection

In conjunction with the selection of the two or more viruses and selection of one or more Subgenomic Viral Replication Systems (SVRS), the host cell is selected from available supplies of cells. While any host-cell may be suitable, particular host cells are desired for broad-spectrum screening based on the viruses and/or Subgenomic Viral Replication Systems (SVRS) to be assayed. In some embodiments, the host cells will be from the same organism that is capable of harboring the virus of interest. It is desirable to select host cells that most closely correspond to the environment of a host cell of a natural viral infection. For example, for a human infecting virus, it is desirable to select a human cell line, and preferably one in which the virus would commonly be hosted. In other embodiments, the host cells that are preferred are any animal cell. In other cases, depending on the particular virus to be assayed, the host cell may be from any eukaryotic family, including plants, fungi, insects, and protists. Additionally, it may be preferred in some embodiments to select a diploid cell line as a host cell. The host cells may be either primary cells or cells replicated from an established cell line.

In the broad-spectrum antiviral method, the selection of the host cell may be independent of the virus or the Subgenomic Viral Replication System (SVRS) or may be dependent on the ability of the virus or Subgenomic Viral Replication System (SVRS) to be harbored in and grown or replicated in the particular host cell. The host cell should provide an effective host environment for assaying and evaluation of the antiviral replication efficacy of the compound to the particular Subgenomic Viral Replication Systems (SVRS). It is also desirable that the host cells grow or replicate under similar conditions such that each cell will support replication of the Subgenomic Viral Replication System (SVRS) that it contains.

The Subgenomic Viral Replication System (SVRS) in the cells may be transient or stably maintained in the host cells for the desired assay period. The desired assay period is the period of time for the particular Subgenomic Viral Replication System (SVRS) to replicate such that an effective and accurate assay results and is sufficient time to enable the evaluation of the antiviral impacts of a compound on viral replication.

It may also be desirable to assay compounds for broad-spectrum antiviral efficacy by utilizing multiple host-cells. In one embodiment one or more different cell lines from human, hamster, mouse, dog and monkey of various cell types and tissues have been utilized in broad-spectrum antiviral screening. In one example, from 108 cell lines, 66 host-cell assays were developed and each was assayed with at least one replicon Subgenomic Viral Replication System (SVRS) representing the viruses of HCV, Yellow Fever, West Nile, Ebola, VEE, Sindbis, and Influenza. A variety of reporter genes were used in this assay. In this example, from the 66 replicons containing cell lines, 5 host cells were identified as being suitable hosts for broad-spectrum screening of the replicons of HCV, YF, WN, Ebola and RSV viruses.

The selection of one or more host cells for broad-spectrum screening of Subgenomic Viral Replication System (SVRS) may also be a function of one or more criteria and factors associated with the particular step of the broad-spectrum screening method. For example, in a primary screen process, host cell selection should be selected based on additional consideration of the amenability of the host cell to automation of the assay process and to host cells that demonstrate superior attachment to the tissue culture plates that enable assay automation. As the reporter gene signal strength may vary from cell type to cell type, selection of a host cell should consider host cells that provide improved or superior signal strength from the reporter gene. To support transient transfection, it is also desirable to select a host cell that demonstrates, higher transfection efficiency such as host cells known in the industry as 293T or Baby Hamster Kidney (BHK). Furthermore, it is also desirable to select a host cell that has demonstrated higher survivability during freezing and thawing cycles which are inherent during the assay and screening process.

For a secondary stage of screening, consideration in the selection of host cells may vary as often the secondary stage is focused on verifying primary assay results. As such, for the secondary screening stage, host cells should be selected that have improved or superior viral assay results. Additionally, during secondary screening, a viral assay may use the live virus rather than a Subgenomic Viral Replication System (SVRS). In such a case, the host cell should permit replication of the virus rather than Subgenomic Viral Replication System (SVRS) replication. As such, host cells selected for the secondary screening may likely be different from the host cells selected for the primary screen. This is understandable as the primary screening stage is often designed around factors that enable high throughput screening with an initial set of one or more Subgenomic Viral Replications Systems (SVRS) which is different that secondary screening which may include additional or different Subgenomic Viral Replication Systems (SVRS) or may include live virus cells, which may not be amenable to the same high-throughput assay process.

Screening Compound Selection

Compounds to be assayed and screened in the broad-spectrum antiviral method are unlimited because the broad-spectrum method provides the opportunity for high-throughput and lower cost assaying of compounds against multiple viruses. As such, a library of broad-spectrum compounds may include one or more commercially available libraries as well as one or more libraries of natural products and synthesized small molecules.

In one embodiment, the selection of compounds to be screened may begin with a review of one or more commercially available catalogues of compounds from commercial suppliers. The selection of one or more commercially available catalogue of compounds may be based on a number of factors including cost, diversity, purity, variety of classes of compounds, prevalence compounds or classes of compounds which may be easily made into drugs for ultimate treatment in a patient. Such catalogues or libraries may include natural product libraries, synthetic libraries, and custom synthetic libraries. After one or more catalogues of compounds have been selected, undesirable compounds such as those with known cardiac problems or compounds that are known to sit in fatty tissue may be removed from consideration to form the broad-spectrum screening compound library. Further fine tuning of the compounds within the screening compound library may be based on expected or anticipated cell-based screening methods or strategies. Further selection of screening compounds may consider the selection of compounds or compound classes with known structure and known impurities or impurity levels. In addition, additionally libraries of compound may be added that contain compounds without structure.

It is also possible to supplement the commercially available compounds with one or more other libraries of compounds. For example, a natural products library/catalog, containing compounds from nature that have been screening and catalogue by a natural products supplier may be added to the broad-spectrum screening compound library.

In one embodiment, any candidate antiviral agent or compound may be evaluated using the broad-spectrum methods including any small to large organic or inorganic molecule, provided the compound is accepted by the host cell when added to the host cell and virus or Subgenomic Viral Replication System (SVRS) culture. For example, to assist in this uptake, the candidate antiviral compound may be formulated into compositions comprising excipients such as liposomes and amphipathic compounds. The host cells harboring a Subgenomic Viral Replication System (SVRS) may also be treated to assist in the uptake of the candidate antiviral compound by treating with polyethylene glycol or with an electroporation device.

In another embodiment, the broad-spectrum library of compounds may be a subset of the full library which may be used at one or more particular stage of the broad-spectrum screening process. For example, one subset library of candidate antiviral compounds include nucleotides, nucleosides, and nucleoside analogs (e.g., ribvirin). Additionally, secondary metabolites and other small chemicals, bioactive amino acids, oligopeptides or polypeptides may be tested for antiviral activity.

In practice, in one embodiment a broad library of commercially available chemicals may be obtained from a commercial vendor. The commercial library is prescreened to identify compound species that may be considered “druggable,” e.g., compound species or classes that have appropriate calculated logp, solubilities, low predicted toxicities, moderate predicted metabolism or other desirable characteristics.

Also in practice, once the compounds have been selected for broad-spectrum screening, they should be prepared for assaying which may involve dilution of a compound to a particular concentration level. For example, each compound within a library may be dissolved to a concentration of 10 mM as a standard concentration or dilution standard. However, for screening additional preparation of the compounds may be required. Each compound may be diluted in a variety of dilutions for screening the various dilution levels of the compound as the antiviral efficacy of the compound may be directly related to the strength of the compound. As such, various dilutions amounts may be screened either initially during the primary screen, or during a secondary or subsequent screening method. For example, for the primary screen all compounds in the broad-spectrum screening library may be diluted to a concentration of 25 uM. As a result of the primary screen, any compound with having greater than 80% antiviral efficacy at the 25 uM concentration level is again assayed at a range of concentrations for antiviral activity and toxicity (including the identification of an EC50/CC50 measurement).

Similarly, any compound having greater than 80% antiviral efficacy at the 25 uM concentration level may be identified as a potential broad-spectrum antiviral compound class or core structure class. In this embodiment, a core structure class may be defined as any class of compounds having at least one member compound with greater than 80% antiviral efficacy at the 25 uM concentration level. In this case, additional compounds of the same compound class may be assayed to identify additionally class members that have greater than 50% efficacy at the same 25 uM concentration level. Of course, this is only one example and other antiviral efficacy thresholds and concentration levels may also be chosen for broad-spectrum screening. For example, in one alternative, a compound class which exhibits an EC50 of less than 10 μM in a primary assay and greater than a 10 Selectivity Index (SI) may be deemed of highest broad-spectrum antiviral importance for further assays and screening. One consideration in establishing these thresholds and cutoffs is the practical feasibility of the testing as the lower the antiviral efficacy level established for the screening, the number of compound classes and compounds to be assayed significantly increases. As such, these variable criteria for the screening of compound for broad-spectrum antiviral efficacy may be adjusted based on the results of the assays and are not biased by the chemistry of the compounds being screened.

The selection of the compounds and natural products within the broad-spectrum screening library is a function of the method of broad-spectrum screening against Subgenomic Viral Replication System (SVRS) which provides for high throughput screening of a large library of compounds against multiple viruses. As such, the broad-spectrum screening library may be very large and therefore may include many compounds and variety of compounds that would not traditionally be included in antiviral screening do to the higher costs and hazards associated with traditional screening methods. This is in contrast to an antiviral drug screening library and a method that focuses on a target and a chemical compound that is considered by chemical composition to be have potential activity against this target, not the viral replication process. In target-based screening, consideration of the number of compounds having antiviral efficacy against one or more viruses is not relevant as target-based screening is focused on screening for antiviral efficacy against a single known and predetermined target. If a compound results in an antiviral activity at or above a predetermined level, that particular compound or compound class is manipulated in an attempt to increase the efficacy of the compound against the target.

In contrast, broad-spectrum screening changes the medical chemistry process of screening compounds or class of compounds. Broad-spectrum screening provides for compound identification of compounds that are active against multiple viruses which may be two or more, or as many as all viruses being screened. For example, in one embodiment, antiviral efficacy is desired for more than 30 different viruses. As such, changing constituents of a compound or within a class of compounds in a broad-spectrum screening method requires that the compound increase its efficacy against multiple viruses at the same time, which is significantly different from simply increasing efficacy against a single virus or target.

Assay Selection

For any particular Subgenomic Viral Replication System, the appropriate assay method and/or system to measure replication of a virus for broad-spectrum antiviral activity is the application of principles and methods which have been tailored and customized to the requirements of screening a substantially large number of compounds against numerous viruses. As such, the antiviral effect of a candidate antiviral compound is determined by assessing the amount of replication that has occurred after the application of the compound and comparing that with a virus which has not received a compound. The difference is indicative of any antiviral effect of the compound on the virus. Examples of methods for making these determinations include any method of RNA or DNA quantification that involves target amplification, such as quantitative RT-PCR or PCR or transcription mediated amplification; or non-amplification method of DNA or RNA quantification, particularly those that involve signal amplification such as branched-chain DNA, but also Northern and/or Southern hybridization, in-situ hybridization, etc. To ascribe copy numbers of a replicon, comparison with RNA standards may be made. The Real-Time Reverse Transcription Polymerase Chain Reaction (rtRT-PCR) assay method provides quantifiable data related to a measurement of antiviral activity of a compound.

In one embodiment, a particular assay method and system may be desirable, if, for example, the candidate antiviral compound was an inhibitor of an enzyme that is essential for viral replication. Any method for quantification of the specific proteins whose level is dependent on viral replication may be useful. Such assays could include for example immunoassays such as EIA, ELISA, immunoblotting, immunofluorescence or immunoprecipitation; assays for enzymatic activity of a particular viral enzyme, for example RNA or DNA polymerase, protease, helicase, thymidine kinase, ribonucleotide reductase, etc. or assays of products of reporter genes that have been fused to a particular viral protein or otherwise inserted into the genome. Examples of these reporter proteins include luciferase, green fluorescent protein, and beta-galactosidase.

One quantified measurement that is a desired output of an assay method is the effective concentration (EC). An effective concentration measurement such as an EC50 provides a standard of measure of the effective concentration that causes for 50 percent of the viral replication observed in untreated controls. EC50 measurements typically range from nanomolar to more than millimolar, with a measured EC50 of less than 10 micromolar indicating good antiviral efficacy. Another quantified measurement desirable of the assay method is the cytotoxic concentration (CC). For example, one standard of measure is a CC50 which is a standard of measure indicating the concentration that causes 50 percent of maximal cytotoxicity. CC50 measurements often range from 0 to over 100 micromolar, with a measured CC50 of greater than 75 indicating low cytotoxicity. Another quantified measurement is a Selectivity Index (SI). The SI may range from 1 to more than 100 with a desired measured SI of more than 10.

In general, broad-spectrum antiviral compound screening and discovery is not directed to one or more targets which may be responsible in one form or manner for one or more functions of the viral replication process. However, the broad-spectrum antiviral method described herein may result in the identification of a target that is common to one or more viruses. Additionally, the broad-spectrum antiviral (BASV) method may be utilized to screen large number of compounds against viruses that have one or more known targets.

In the cell-based broad-spectrum antiviral method, when one or more Subgenomic Viral Replication Systems (SVRS) are utilized, antiviral inhibitors of any biochemical pathway involved in viral genome replication and transcription of viral genes may be identified. To accomplish this, the assay method and screening procedure is selected to enable evaluation of the effects of a compound on the pathway of interest to be able to measure the effects of a candidate antiviral compound on that pathway. For example, to identify a compound that targets any pathway involved in replication, an end product of replication (for example the replicated genome) is measured after treatment with the compound by, e.g., performing quantitative PCR to quantify the amount of a representative portion of the genome that is present. Alternatively, to identify a compound that targets a specific pathway involved in replication, e.g., translation of RNA polymerase, the assay method should measure the particular component. For example, an antibody assay may be used to quantify the RNA polymerase.

Traditional antiviral compound screening methods have utilized biochemical assays as the primary screening method. In these methods, a cell-based assay is performed as a secondary assay only after a lead compound has been produced by the primary screening method. As such, quantifiable data resulting from a cell-based assay is not developed until the second stage of screening and therefore is not available to aid in the identification of the lead compound.

For broad-spectrum screening, assay selection may be based on factors related to screening for antiviral compounds in the broad-spectrum antiviral screening format. A first factor to be considered is the selection of an antiviral assay that is capable of covering the wide range of viruses which are to be included in the assay, many of which are from a diverse group of virus families. The range of diversity between individual virus families to be assayed may significantly favor one assay method over another assay method. In broad-spectrum discovery, it is fundamental that the assay method provide the ability to discover an inhibitor of viruses from different families or within a given family.

A second factor in the selection of an assay method for broad-spectrum screening is the ability to create assays for a given virus based on the selected host cell line's ability to support viral replication. For example, a particular parental cell line may be selected for development of a viral replicon but later it may be discovered that a specific replicon is cytotoxic to the host cell. Assay selection is a function of whether or not a given cell line constitutively supports replication of a viral replicon. Toxicity can be both acute and chronic in nature. Acute replicon toxicity would cause the host cell to essentially “die.” Chronic replicon toxicity may cause the host cell to inhibit or significantly reduce replication of the replicon.

A third factor is the ability to mix and match (multiplex) the viral screening assays. For example a scientist may develop an assay using a different reporter gene (which is a quantitatively measured amount of a virus in a cell). Reporter genes are amino acid sequences which code for the translation of specific protein, such as renilla luciferase (RLuc) or beta-galactosidase (β-gal). For example, the RLuc reporter gene may be used to develop the West Nile and the Yellow Fever replicon systems.

In one embodiment, a reporter gene assay may provide for a substantial reduction in the cost per assay for the many assays that are required for broad-spectrum screening. By using a reporter gene assay, a cost of less than $0.25 per well has been achieved for broad-spectrum screening using the present method. Additionally, a reporter gene assay provides an increase in the speed of assay processing and therefore a reduction in the time required for each assay. This results in the ability to screen a substantially greater number of compounds against a greater number of viruses, host-cells, and concentration levels than otherwise possible. Additionally, reporter gene screening provides for broad and dynamic linear readout of the assay which improves the overall quality and ability to quantify the results of the assay. However, the reporter gene based antiviral screening system does not directly measure the amount of the viral target RNA. Where such a direct measurement is desired, a quantitative reverse transcription-polymerase chain reaction (qRT-PCR) assay may be utilized either in place of or as a supplement to the reporter gene assay.

As introduced above, the quantitative reverse transcription-polymerase chain reaction (qRT-PCR) assay provides for a direct readout or measurement of the amount of the viral target RNA. Additionally, qRT-PCR assays may provide for an increased breadth and dynamic linear range of the assay. However, qRT-PCR assays are costly and may result in a cost per well of $5 or more. Additionally, qRT-PCR assays require multiple steps during the assay process or protocols and take considerably more time, and therefore are comparably very slow.

In an alternative embodiment, broad-spectrum screening may optionally utilize a multiplexed assay method to reduced costs and reduce the time required for the screening of the many compounds and the many viruses. The multiplexed assay method provides the ability to separately analyze the antiviral effect of a compound on multiple viruses simultaneously or sequentially, but based on a single assay well containing the multiple viruses. In a multiplexed assay, two or more different viruses or Subgenomic Viral Replication System (SVRS) cells are placed into a single well and treated with a single compound. Each marker or reporter gene for a Subgenomic Viral Replication System (SVRS) generates its own signal indicative a viral replication or presence. Each of the different signals generated from these replicons within a single well are then analyzed. To distinguish activities from each individual replicon, related to each virus, different markers or reporter genes should be constructed for use simultaneously within each well. As one example of a multiplex assay method, a multiple subgenomic replication culture (MSRC) which is also referred to as a mixed viral replicon culture (MVRC) may be employed. The MVRC method is described in U.S. patent application Ser. No. 10/060,941, entitled MULTIPLE VIRUS REPLICON CULTURE SYSTEM, which is incorporated herein by reference.

As an example, in one multiplexed assay, the different reporter genes for each Subgenomic Viral Replication System (SVRS) provide different fluorescent end product (using e.g., molecular beacons specific for each viral RNA, or different fluorescent proteins and/or fluorescent products of an enzyme encoded by the Subgenomic Viral Replication System (SVRS). Each cell is analyzed by quantifying the intensity of the fluorescence of the fluorescent moiety associated with the Subgenomic Viral Replication System (SVRS) in that cell. Optionally, such data is obtained from each cell, e.g. using a fluorescence activated cell sorter.

A multiplexed assay for broad-spectrum antiviral screening also permits detection of antiviral compounds that would be overlooked in one or multiple single virus screening systems. A multiplexed assay may enable the identification of both specific inhibitors that target any one of the multiple targets and broad-spectrum compounds that affect multiple viruses may be identified. Simultaneous recovery of viral inhibition data for a variety of distinct viruses or Subgenomic Viral Replication Systems (SVRS) permits identification of antiviral compounds that might otherwise be overlooked.

Use of a multiplexed viral assay in broad-spectrum screening further permits the desired quantification of antiviral activity through the use of assay methods including quantitative reverse transcription-polymerase chain reaction (qRT-PCR) mediated detection. Additionally, molecular beacon-based hybridization probes have also been employed to detect multiple viruses in a single sample. Broad-spectrum antiviral screening may also utilize multiplex assays for distinct gene reporters that may be independently assayed from the contents of a single culture well. Additionally, broad-spectrum antiviral screening methods employ one or more Subgenomic Viral Replication Systems (SVRS).

A multiplexed assay may contain one or more viruses, one or more Subgenomic Viral Replication Systems (SVRS) or a combination of the two. However, multiplexed assays require that the effect of the candidate antiviral compound on each individual Subgenomic Viral Replication System (SVRS) be independently discernable or evaluated uniquely which, as discussed above, may require the selection of viruses or SVRS's with separate and distinct reporter genes.

Additionally, a multiplexed assay may be used to mix viruses or Subgenomic Viral Replication Systems (SVRS) representing different viral species, families, subfamilies, or subtypes in order to confirm the broad-spectrum antiviral activity of a compound. In another embodiment, a multiplexed assay may be used to analyze and evaluate different reporter genes when multiple Subgenomic Viral Replication Systems (SVRS) representing a single virus are assayed. In this case, each Subgenomic Viral Replication System (SVRS) of the virus is developed to utilize a different report. Additionally, in such an assay, the sum effect of each reporter gene on the replication of the multiple Subgenomic Viral Replication System (SVRS) representing the single virus may be evaluated.

Broad-Spectrum Antiviral Screening

According to one or more embodiments, the broad-spectrum screening method identifies one or more compounds or classes of compounds that demonstrate broad-spectrum antiviral activity against two or more viruses. As noted, broad-spectrum antiviral screening utilizes cell-based screening and discovery and does not utilize target-based screening and discovery. Such cell-based screening provides that ability to screen significant numbers of compounds for antiviral activity against multiple viruses or Subgenomic Viral Replication Systems (SVRS) some of which are difficult or impossible to effectively and efficiently be screened. Additionally, each compound may be screened at a variety of concentration and against a variety of virus/host cell combinations. For example, in one embodiment a primary screen may include screens against HCV using an immunoassay for NPTII, RSV using an enzymatic assay for β-gal, WNV using an enzymatic assay for RLuc, YFV using an enzymatic assay for RLuc, and EBOV using an enzymatic assay for RLuc.

In one embodiment of the broad-spectrum method one or more assaying or screening steps may be performed to provide quantitative data which is analyzed to determine broad-spectrum antiviral activity for a compound or for a class of compounds. In a subsequent screening step, it may be desirable to determine the cytotoxicity of a compound or class of compounds in order to determine the effect of the compound on one or more host cells. Additionally, compounds are assayed initially against a subset of the total desired viruses or a subset of the potential compounds. For example, in a primary screen a subset of compounds may be a representative of compounds for a select subgroup of compound classes. In a further screening step, a different assay method may be desired or required for the determination of optimal assay timings. In this case, it may be desirable for each assay to determine the optimal time period for freezing cells, for adding compounds to the host cells, for evaluating the effects of the compounds on the replication, for identifying the maximum replication signal strength, and the earliest time point for addition of a compound after thawing of the host cell.

In one embodiment, an initial or primary screening step or stage may include infection-independent screening of viruses by using Subgenomic Viral Replication Systems (SVRS) with high-throughput testing with a large number of compounds from a number of classes of compounds. In other embodiment, a mixture of viruses and/or Subgenomic Viral Replication Systems (SVRS) may be used or all viruses. As an example, a single Subgenomic Viral Replication System (SVRS) may be selected as a prototype for screening based on a function of reliability, signal strength, and correlation. In such an embodiment, an entire library of compounds or a subset thereof may be screened against the selected prototype Subgenomic Viral Replication System (SVRS). For example, assays may be performed using high-throughput assay methods utilizing more than 100,000 compounds representing all compound classes within a particular compound library.

A subset of compounds may be selected after this initial screening stage against a single virus. The subset may be comprised of compounds demonstrating the highest levels of efficacy against the one virus, or an efficacy greater than a predetermined efficacy. For example, a priority may be given to the compounds demonstrating the top 1, 2 or 5 percent of antiviral efficacy or those with greater levels of antiviral efficacy against the greater number of viruses.

The subset of compounds can be screened using two or more additionally viruses or Subgenomic Viral Replication Systems (SVRS) and one or more host cells. Once these additional two or more viruses are assayed, the quantifiable data obtained from the assays, (such as EC50, CC50 and SI) are recorded and may be ranked or rated. Each of these compounds may be sorted and ranked by demonstrated antiviral efficacy against the two or more viruses and/or Subgenomic Viral Replication Systems (SVRS).

Whether or not these optional or additional screening steps are performed, compounds are again rated or ranked to identify the compounds with the highest priority representing broad-spectrum antiviral activity. As an optional rating and selection method, preference may be given in the rating and selection process to compounds that exhibit a QSAR that is the same for two or more viruses. In such cases, having equal QSAR ratings may be indicative of antiviral activity of a compound against a compound target associated with each of the two or more viruses.

When the antiviral activity of the candidate antiviral compound is measured by determining whether replication has been inhibited, the cell culture is incubated for a period of time sufficient to allow a measurable amount of replication to occur, but not so long that the cells overgrow the culture dish thereby making it impossible to evaluate. For example, the inventors have determined that for some virus/cell combinations, a 20 to 24 hours incubation is optimum. This includes virus/cell combinations such as: hepatitis C virus replicon in human hepatoma (Huh7) cells or hepatocytes; Sindbis virus replicon in baby hamster kidney (BHK) fibroblasts; yellow fever virus replicon in Huh7 cells; and respiratory syncytial virus (RSV) minigenome transiently expressed in the BHK cells harboring a Sindbis replicon.

The broad-spectrum antiviral screening method of the present invention may perform a primary screen of compounds against two or more viruses and may include one or more Subgenomic Viral Replication Systems (SVRS). A representative process includes the selection of a representative virus to be used in the primary screen. This process includes the selection of a virus or Subgenomic Viral Replication System (SVRS) representative of one or more viruses and the selection of their host-cells. For example, the selected viruses or Subgenomic Viral Replication Systems (SVRS) may include one virus representative of each of the major families of viruses. A live virus may also be used in the primary screen when a low bio-safety level virus is available such as a BL1 or BL2. The selection of the representative virus or Subgenomic Viral Replication System (SVRS) may be based on factors such as availability, reliability, ease of handling.

Next, the compounds from within the entire library or a subset of the entire library are selected to be individually screened against each virus or in a multi-virus screening method. Additionally, the various concentration levels for the compound are selected. The reporter genes are assayed in order to quantify the viral replication or antiviral activity of each compound. Included in this process is the normalization of the cytotoxicity to ensure effective and consistent measurement of cytotoxicity of each compound or compound class.

In another embodiment, a secondary screening step may be performed to further screen and refine the compounds to identify those compounds demonstrating the best broad-spectrum antiviral efficacy. A secondary assay may be performed against different viruses and/or different Subgenomic Viral Replication Systems (SVRS), one or more of which may be representative of the same virus. For example, a second screen may include infection-dependent screening of BL2 viruses which may require the utilization of a medium-low throughput assay method rather than a high-throughput and automated assay approach used in the primary screening step, if applicable. In one embodiment a second screen may be performed using Subgenomic Viral Replication Systems (SVRS) of prototype BL2 with two or more viruses of other virus families, one or more of which may be a BL2 rated virus. In another embodiment, a secondary screen may include a confirmation of antiviral activity against viruses within the same family as the BL2 prototypes, e.g., other BL2s.

A secondary screen may also be performed on the best candidate compounds from the primary screen. After a rating of the compounds during the primary screen, compounds rated with the highest broad-spectrum antiviral efficacy are selected for a secondary screening. For example, the selection of the top 1.0 percent or the top 0.1 percent may be selected for antiviral efficacy against one or more viruses or Subgenomic Viral Replication Systems (SVRS) in a secondary assay. Furthermore, the chemical classes of the compounds may themselves be prioritized when and where two or more compounds demonstrate higher levels of broad-spectrum antiviral efficacy which may be indicative of a compound class that needs further study. During the secondary assay stage one or more of EC50, CC50, and SI may also be determined and the compounds may again be sorted, rated and ranked.

In another embodiment, a secondary assay or screening may include Subgenomic Viral Replication Systems (SVRS) of additional or other highly hazardous viruses if these viruses where not assayed in the primary screening phase. For example, viral assays for BL2, BL3, and BL4 viruses for one or more family of viruses may be assayed during a secondary assay to provide further data and aid in the identification of broad-spectrum antiviral efficacy.

Additionally, a different assay method may be used in a secondary screen to provide an alternative perspective and analysis. For example, a recombinant/reporter gene assay may be used for prospective broad-spectrum antiviral compounds during the secondary assay stage.

In another embodiment of the broad-spectrum antiviral screening method, a secondary screen is performed against viruses or Subgenomic Viral Replication Systems (SVRS) representative of all desired families of viruses if not performed during the primary screen. In such a case, the secondary screen is intended to provide quantification of the broad-spectrum antiviral efficacy against all of the desired family of viruses. Additionally, in the secondary screen, the same or additional assays of the compounds against the viral assays may be preformed. For example, reporter assays, RNA-based assays, and viral assays may be performed during the secondary broad-spectrum secondary screen. Additionally, while Subgenomic Viral Replication Systems (SVRS) may, in one embodiment, be desired for the primary screen, during the secondary screen, it may be desired that the live virus itself be used in the assay. This may be desirable in order to address any potential issues related to differences between the representative of the Subgenomic Viral Replication System (SVRS) and the infectious virus. However, as noted above, for many viruses this option may be limited due to the hazardous status of the virus as a pathogen.

As with the primary screen, quantifiable data is produced during the secondary screen for broad-spectrum antiviral compounds. These may include the EC50, CC50, and SI measurements as well other measurements indicative of broad-spectrum antiviral activity and toxicity of the compound. The secondary assay or screen may provide for a direct measurement of the efficacy of a possible lead compound against each available Subgenomic Viral Replication System (SVRS) in order to further determine the broad-spectrum antiviral properties of the compound.

In another embodiment, the broad-spectrum antiviral screening and compound discovery method may include further assaying or screening steps to further refine the broad-spectrum antiviral lead compound or lead compound class identification and selection identified in a primary and secondary screen. For example, it may be desirable to perform an additional screening step which includes assays against the live viruses themselves where virus or infectious screening was not performed in earlier screens. A virus screen may be performed against the virus itself to verify antiviral activity. However, because the virus itself is being used, this process may be slow and costly especially if performed against highly hazardous viruses such as viruses rated at a bio-safety level of BL3 and or BL4. At this stage, however, the earlier screening processes will likely have significantly narrowed the compounds from the potentially more than 100,000 compounds with numerous compound classes to less than one percent of compounds, and maybe less than 0.01 percent (or 10 compounds) to be assayed against the most hazardous, difficult and costly viruses such as HIV-1.

In another embodiment, the significantly reduced group of compounds may be tested in live or animal models to determine antiviral efficacy of the infectious disease associated with the virus. This may be required when the virus does not replicate well in a laboratory setting, such as HCV. It may also be desirable to test all viruses rated as BL2, BL3, and BL4, or a combination or subset thereof to identify potential broad-spectrum antiviral compound and/or identify or verify a target common to more than one virus.

To verify broad-spectrum antiviral efficacy, it would be desirable to perform tests or assays against the live virus in a well or a host animal to confirm antiviral activity against two or more of the BL3 and/or BL4 viruses, and may be desirable to include a BL3 or BL4 from each family of virus to potentially identify a strong broad-spectrum antiviral compound.

In one embodiment, the screening process may utilize: sequential assays of single virus screening systems. In standard single-virus screening systems where antiviral compounds affecting a single virus are generally identified by an antiviral effect above a threshold inhibition level. In those procedures, antiviral compounds with sub-threshold levels of activity against the screened viral target are discarded.

In one embodiment of the broad-spectrum antiviral screening method, once a potential lead compound is identified as a potential broad-spectrum antiviral, the broad-spectrum antiviral efficacy is verified. The broad-spectrum antiviral efficacy measured in the initial and secondary is confirmed or verified utilizing a confirmation assay which may be a different assay method than utilized in either the initial or secondary assays or may be a repeat of one of the earlier assays. For example, a viral yield assay or qRT-PCR assay may be used.

In another embodiment, the protocol and process for verification of broad-spectrum antiviral efficacy may include a review of the reporter genes or reporter gene assay. In another embodiment, it may be desirable to quantify a decrease in the Subgenomic Viral Replication System (SVRS) and to identify any changes to a cellular house keeping gene.

During any verification step, it may be desirable to ensure that the observed antiviral efficacy is not simply a killing of the host cell by the compound. In other words, the cytotoxicity of the compound should be within a predetermined range.

In practice, after adding a candidate antiviral compound to a cell culture containing the host cell and one or more viruses or Subgenomic Viral Replication Systems (SVRS), the cells are incubated under conditions and for a time sufficient to detect an antiviral effect by the compound. Depending on the virus and or Subgenomic Viral Replication System (SVRS), the reporter gene, and the nature of the host cells, the optimum time of incubation could be anywhere from 1 minute to 48 hours, or longer. The shorter time periods are envisioned, for example, where the antiviral activity of the candidate antiviral compound is measured by evaluating whether the compound binds to, e.g., a particular protein, such as an enzyme, where inhibition of enzyme activity, or binding of the candidate compound to the enzyme, is measured.

As noted, the broad-spectrum antiviral screening process is cell-based not target based. As such, the virus and/or Subgenomic Viral Replication System (SVRS) are evaluated in one or more of the possible set of host cells. For example, as discussed above, one embodiment of the broad-spectrum antiviral method includes screening of the multiple Subgenomic Viral Replication Systems (SVRS) against one or more of cell lines. To aid in the process of screening up to 100,000 compounds against two to 30 or more Subgenomic Viral Replication Systems (SVRS) in up to 108 cell lines, it is suggested that an APC number and/or bar code be assigned to each cell line with each Subgenomic Viral Replication System (SVRS), and to each assay.

In the broad-spectrum method, it may also be desirable to verify and test the reliability of replication signals. Such reliability may be tracked and evaluated for each plate (96 assay wells), between plates (24 wells), and between days. Verification may include blind spike experiments and dose response experiments.

Each is compound, Subgenomic Viral Replication System (SVRS), and cell line is tracked and maintained as a function of the multiple assay and assay results where efficacies against two or more viruses or Subgenomic Viral Replication Systems (SVRS) are observed. In broad-spectrum antiviral screening, groups of plates are reviewed. In broad-spectrum antiviral screening, each batch of cells is the same.

To aid in broad-spectrum antiviral discovery and development method, multiple viruses may be assayed at the same time in a multiplexed manner to determine broad-spectrum antiviral efficacy of the compound. Tracking is therefore required for multiple viruses and multiple assays which is considerably different from tracking assays for a specific or single activity against a single virus or target. Every compound is checked, labeled, and data entered for each assay for each and every compound.

Also for the broad-spectrum antiviral method, quantified antiviral data is developed for all compounds and all assays and multiple representatives for all viruses at an early stage of the screening process. Every assay goes through the same steps so that the quantifiable data is developed for each compound for each assay, for each Subgenomic Viral Replication System (SVRS) as an output to each and every stage of the screening process. This is different from target-based screening. In target-based screening, EC50 quantification is performed after lead-compound selection to determine and quantify efficacy against the specific virus. As such, the broad-spectrum antiviral method results in antiviral quantitative efficacy data during the broad-spectrum method when such data is not available for screening during target-based screening and lead development.

Broad-Spectrum Antiviral Data Analysis and Compound Identification

The screening for broad-spectrum antiviral activity provides a new approach to screening including the data collection and analysis phases. During the broad-spectrum assay process, one or more quantifiable antiviral data is obtained that reflects a quantifiable measurement of the antiviral activity or efficacy of the compound against each tested or assayed virus. Examples of such quantifiable data include an EC50 measurement, a CC50 measurement, or a Selectivity Index measurement. This antiviral quantitative data is recorded for each and every compound, against each and every virus, in each and every assay, resulting in substantial data. All of this data is available for review to identify determine broad-spectrum antiviral indications or trends.

In contrast to prior methods, the quantified data from the cell-based screening is readily available for review and the identification of candidate broad-spectrum antiviral compounds or classes of compounds, or to move to the next screening stage may be based on a ranking and prioritization of the quantitative data for antiviral activity of each viruses against the two or more viruses. Additionally, the broad-spectrum method provides detailed quantifiable data that is uniquely available for review for quality control and monitoring of each and every assay, the quality of the host cells, the quality of the virus or subgenomic viral replication system (SVRS) cells, and the quality of each and every screening plate. Each of these may be reviewed to evaluate the quality and effectiveness of the assays and therefore as a means of validating the assays and the rating and ranking of each compound. Also, such detailed data provides a considerable source for the selection of target-based research and the identification of virus-specific compounds for virus-specific drug development.

In one or more embodiment, the broad-spectrum antiviral compound screening and discovery method described herein may be adjusted to consider any and every factors that may be later identified as relevant to antiviral drug development. For example, if it is discovered that a potential single target is being inhibited by the compound among the two or more viruses, consideration may be give to determining whether the target has an equal or different importance for the replication of each of the viruses. If there is a different importance between two viruses, additional assays may be required or other steps taken to adjust the quantifiable data or test results so that the assay results are “standardized” between the two viruses to reflect that the compound is inhibiting the same target in each virus. In another case, where the target between two viruses is the same protein family, it may be necessary to determine whether the two or more viruses use the same or different family members which may negatively impact the assay results. In yet another case, where there are multiple targets and different targets for each virus, it may be necessary to determine the particular targets being inhibited by each compound because the antiviral assay may be skewed as they may be representative of a summation of multiple weak antiviral activity against multiple targets, rather than a strong antiviral activity against a single target. As such, the assay results may appear to have similar effects between two viruses, but in fact, reflect antiviral activity due to different mechanisms or targets.

The broad-spectrum rating method and system may be based on any set of quantifiable antiviral data including the data generated during antiviral assays. For example, where no other quantifiable antiviral data exists, the quantified data from the broad-spectrum screening method may include measurements or quantified data for each compound, at each dilution level, for each virus or each subgenomic viral replication system. As discussed above, this data may include a measurement of the antiviral efficacy as measure by the EC50, CC50 and SI measurements. EC50 is the concentration at which an inhibitor decreases the replicon reporter signal to 50% of the control value. CC50 is the concentration at which an inhibitor causes the toxicity assay signal to be decreased to 50% of the control value. SI is the Selectivity Index or the range between an inhibitors efficacy and toxicity. These or other quantifiable measures may be utilized to rate and rank each compound for broad-spectrum antiviral efficacy. The broad-spectrum antiviral rating method rates each compound on their demonstrated quantifiable antiviral efficacy (EC50, CC50 and SI values) from multiple broad-spectrum antiviral screening assays against multiple viruses.

For each assay of each compound for each virus, in one embodiment a total possible points achieved by the compound in one antiviral screening assay may be 1,000 points. The 1,000 points may be divided between the EC50 which receives 200 possible points, the CC50 which receives 300 possible points and the SI which receives 500 possible points as shown as one example in Table 2.

TABLE 2
Broad-spectrum Rating Components for each compound against each virus

| EC 50 | | CC50 | | SI | |
|---|---|---|---|---|---|
| Equal to or < 2 | 200 | Equal to or > 75 | 300 | Equal to or > 50 | 500 |
| 2 to 4 | 165 | 75 to 60 | 248 | 40 | 450 |
| 4 to 8 | 130 | 60 to 45 | 195 | 30 | 400 |
| 8 to 16 | 95 | 45 to 30 | 143 | 20 | 350 |
| 16 to 20 | 60 | 30 to 15 | 90 | 10 | 300 |
| Equal to or > 20 | 25 | Equal to or < 15 | 37.5 | 7.5 | 250 |
| uM Concentration | Assigned | uM Concentration | Assigned | 5 | 200 |
| Range | Points | Range | Points | 2.5 | 150 |

| | | | |
|---|---|---|---|
| Active EC50 Equation | Active CC50 Equation | Egual to or < 1 | 100 |
| y = −49.923 x + 212.200 | y = 3.575 x + 2.802 | | Assigned |
| | | | Points |

Active Equation
y = 100.831 x + 57.0351

The broad-spectrum antiviral rating method and system for candidate compounds provides a higher weight to compounds exhibiting antiviral activity against a greater number of viruses and may penalize a compound exhibiting antiviral efficacy against few numbers of viruses. In one embodiment, as shown in Table 2, each assay of each compound against each of the two or more viruses provides quantitative data for the EC50, for the CC50, and for the therapeutic index. As shown in the embodiment of table 2, a total possible score of 1,000 is allocated to each of these antiviral measurements. In this example, the possible 1,000 point score is allocated with 200 points for the EC50, 300 points for the CC50, and 500 points to the SI. A score for each of the EC50, the CC50, and the SI is determined and the three scores are summed to develop a total score for each particular compound for each virus for which each compound is assayed.

As an example with regard to Table 2, an assay of a first compound may result in antiviral quantifiable data against a first virus with an EC50=3, a CC50=59, and a SI=28, thereby resulting in a score for the first compound against the first virus of 165+195+350=710. That same first compound may result in antiviral quantifiable data against a second virus of EC50=5, CC50=74, and SI=10, which would result in a score of 130+248+300=678. In one example, the first compound may result in scores against five viruses of 710, 678, 625, 680, and 605. In a similar manner, a second compound may result in scores against the same five viruses of 310, 435, 928, 254, and 840. As such, a rating table or assignment method such as illustrated in Table 2 may produce antiviral efficacy ratings for each assayed compound based, on all viruses assayed.

In order to rate the components for broad-spectrum antiviral efficacy, a total weighted broad-spectrum score for each compound is determined that includes that additional weighting factor demonstrating a compound's antiviral efficacy against multiple viruses. As such, a compound demonstrating higher antiviral efficacy against a fewer number of viruses may result in a lower broad-spectrum antiviral rating. There are various methods to accomplish such a rating. One embodiment would be to establish a minimum qualifying value for a compound against each virus. Those scores could be summed with an added weight factor given to each additional virus for which a compound has efficacy in excess of the minimum qualifying value. If a compound does not demonstrate the minimum qualifying value, the compound received zero points toward the broad-spectrum antiviral rating.

For example, as described above, one example of a minimum qualifying value could be 600 points. Per the above example, the first compound with scores against 5 viruses of 710, 678, 625, 680, and 605 results in antiviral efficacy above the minimum qualifying value for all five viruses. By comparison, the second compound, while having two scores 928 and 840 that are greater than any of the scores for the first compound, would actually result in a lower broad-spectrum antiviral score because the compound only demonstrated antiviral activity in excess of the minimum qualifying value against two of the five viruses. As one example, the broad-spectrum rating could be determined by summing the scores in excess of the minimum qualifying value resulting in a broad-spectrum score for the first compound of 3298 and a score for the second compound of 1768. As such, the first compound has a higher broad-spectrum antiviral rating than the second compound. The first compound would be selected for further broad-spectrum screening or as a broad-spectrum lead compound for drug development over the second compound. This is different from target-based screening which seeks to identify compounds with the highest levels of antiviral efficacy against a particular virus and ignores compounds that have lower levels of efficacy against the particular virus and demonstrates broad-spectrum antiviral efficacy against other viruses.

This ranking for antiviral efficacy is different from target-based screening that result in identification of one or more lead-compounds that demonstrate high levels of efficacy. In broad-spectrum antiviral screening, a high level of efficacy against a single virus is not indicative of broad-spectrum antiviral efficacy. In fact, a compound having extremely high efficacy against a single virus and low efficacy against all other viruses will be rejected or ignored by the broad-spectrum antiviral method.

In the broad-spectrum method, other rating and weighting formulas, factors and criteria, while not discussed in the above example, are also anticipated and consistent with the present invention. In practice, such a broad-spectrum rating method or system is implemented in a database or a spreadsheet as an application within a computer system having a processor, input/output device, and a memory. While one or more compounds may be used to implement the above rating and ranking example for broad-spectrum compound identification, it is expected in practice that scores and ratings would be developed from thousands of assays using thousands of compounds against two or more viruses as discussed above. For example, in one embodiment, 100,000 compounds are assayed against 30 viruses and/or subgenomic viral replication systems representative of 30 viruses, or any combination thereof. Additionally, in one embodiment 10 different dilutions of the 100,000 compounds are assayed. In practice, each assay would produce three antiviral measurement data points. As such, a broad-spectrum antiviral screening would produce 90 million data points for analysis. After rating and ranking, the compounds for broad-spectrum antiviral activity, further broad-spectrum antiviral assays may be performed to verify results or one or more lead compounds may be moved to the drug development stage.

In an alternative embodiment, a compound may be selected as a potential broad-band antiviral compound or class of compounds when it demonstrates antiviral activity of an EC50 less than 10 uM and a SI greater than 10 for each virus assayed or a subset of the viruses assayed. To determine broad-spectrum antiviral efficacy, a preference, higher priority, or additional weight is given to a compound or class of compounds that demonstrate levels of efficacy in excess of any predetermined threshold activity level for two or more of the assayed viruses. For example, in the above example, a predetermined threshold antiviral activity level on the 1,000 point scale may, after an initial screening of all compounds, be established between 500 and 1,000 and in one embodiment may be established at 600. After such a process, one or more broad-spectrum lead antiviral compounds or classes of compounds may be selected as a function of the broad-spectrum antiviral rating. For example, all compounds demonstrating a broad-spectrum antiviral rating greater than a predetermined broad-spectrum lead compound level may be selected as a lead-compound or the compounds may be ranked and the top percent or top numbers of compounds may be selected as lead-compounds.

In an alternative embodiment, all compounds with an antiviral efficacy greater than a predetermined level, for example an 80 percent efficacy, may be sorted into compound classes. Each compound class may be quantified or rated for broad-spectrum antiviral efficacy using one or more techniques includes a structure-activity relationship (SAR) and/or a quantitative structure-activity relationship (QSAR) method which identify one or more activity related to one or more structures that are related to the class of compounds. Each of these compound classes may then be prioritized based on such factors as synthesizability, flexibility, patentability, activities, toxicities, and/or metabolism. In this case, all or an additional set of compounds within each particular compound class is assayed and analyzed. As some compound classes may be very large, a subset of the compounds in the classes may be assayed and analyzed and if the class continues to demonstrate broad-spectrum antiviral efficacy in excess of a predetermined level, the remaining members will be assayed. Additionally, where a class of compounds demonstrates desirable broad-spectrum antiviral efficacy, additional members of the class may be obtained or derived and added to the library of compounds to be tested. In such a manner, not only are compounds rated and ranked, but classes of compounds are rated and ranked to identify classes of compounds having broad-spectrum antiviral efficacy.

Broad-spectrum antiviral efficacy may be antiviral efficacy against two or more viruses from a number of different groups of viruses. In various embodiments, broad-spectrum antiviral efficacy may be efficacy one or more combinations of viruses including, but not limited to, antiviral efficacy against two or more RNA viruses, two or more RNA reverse transcribing viruses, two or more RNA retroviruses viruses, two or more double strand RNA viruses, two or more DNA viruses, two or more DNA reverse transcribing viruses. The two or more viruses may be two or more viruses within any single of these virus groups or may be two or more amount two or more of these virus groups.

For example, in one embodiment broad-spectrum antiviral efficacy may be antiviral efficacy of two or more RNA viruses, which may be two or more positive-strand RNA viruses, such as two or more viruses from a group consisting of Sindbis virus, rubella, hepatitis C virus, West Nile virus, yellow fever virus, tick-borne encephalitis virus, Japanese encephalitis virus, coxsackivirus, enterovirus, hepatitis A virus, SARS, astrovirus, Dengue fever virus, poliovirus, and Venezuela encephalitis virus, WEE, EEE, Marayo O'nong nong, Ross River, Chikungunya, DV, Rhinovirus, Feline, murine, Norwalk, Bovine, and human coronaviridae.

Additionally, in another embodiment broad-spectrum antiviral efficacy may be two or more viruses are negative-strand RNA viruses. As one example, the two or more negative-strand RNA viruses may be RSV, Ebola, and Influenza. As another example, broad-spectrum antiviral efficacy may be antiviral efficacy against two or more negative-strand viruses such as respiratory syncytial virus (RSV), Ebola virus, rabies virus, Lassa fever, Argentine hemorrhagic fever virus, La Crosse virus, Rift Valley fever, Hantaan virus, California encephalitis virus, influenza virus A, influenza virus B, measles, mumps, Marburg virus, Bolivian hemorrhagic fever virus, Crimean-Congo virus, HPIV, HMPV, Nipah, Hendra, VSV, LCMV, Junin, Bunyamwera, Uukuniemi, and CCHF.

As another embodiment, broad-spectrum antiviral efficacy may be antiviral efficacy against two or more of HIV-1, HIV-2, HTLV-1, and HTLV-2.

In yet another embodiment, the two or more DNA viruses may be two or more viruses such as human parvovirus, adeno-associated virus, herpes simplex virus type 1, herpes simplex virus type two, human herpes simplex virus type six, human herpes virus type seven, human herpes virus type eight, human adenovirus, BK virus, human papilloma virus, Epstein-Barr virus, JC virus, human cytomegalovirus, and varicella-zoster virus.

In an alternative embodiment, broad-spectrum antiviral efficacy may be antiviral efficacy against two or more viruses of hepatitis C virus, yellow fever virus, respiratory syncytial virus, Sindbis virus, influenza virus A, Venezuela encephalitis virus, West Nile virus, and Ebola virus. In an additionally alternative, it may be antiviral efficacy against respiratory syncytial virus and hepatitis C virus, or against two or more viruses of West Nile virus, yellow fever virus, Sindbis virus, Venezuela encephalitis virus, and Ebola virus.

In another embodiment, broad-spectrum antiviral efficacy may be antiviral efficacy against two or more viruses of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

In practice, the broad-spectrum screening method may be performed in a number of manners. As one example, in one embodiment the inventors have implemented a three-step broad-spectrum screening and discovery method. In step one, a single virus member from each of 6 negative-strand RNA virus families was chosen as a prototype. A Subgenomic Viral Replication System (SVRS) in the form of a minigenome for one of the prototype viruses rated as a bio-level 2 (BL2) was selected and assayed using a high-throughput assay method against an entire library of compounds. Step one included both a primary screen as well as a secondary screen which assayed the five other viruses using Subgenomic Viral Replication Systems (SVRS). From these assays, compounds and classes of compounds were rated and ranked to identify broad-spectrum antiviral candidate compounds and compound classes.

In a second step, low throughput viral assays are performed on the minigenome systems of the prototype BL2 virus and on four additional prototype viruses of the five additional negative-strand virus families. The antiviral activity against the viruses of the original viral family are assayed to confirm the antiviral activity. A quantitative structure-activity relationship (QSAR) assessment was performed for each compound and class of compounds and the compounds and classes of compound are prioritized for broad-spectrum antiviral activity. The leading candidate compounds and classes of compounds were used for a third step of assaying and screening. In the third step, a bio-safety level 4 (BL4) virus was selected for each of the six negative-strand RNA families of viruses. Low throughput assays were performed using animal models with the BL2 and BL4 viruses. From this process, compounds and classes of compounds with the highest rating for broad-spectrum antiviral efficacy will be identified and prepared for broad-spectrum antiviral drug development.

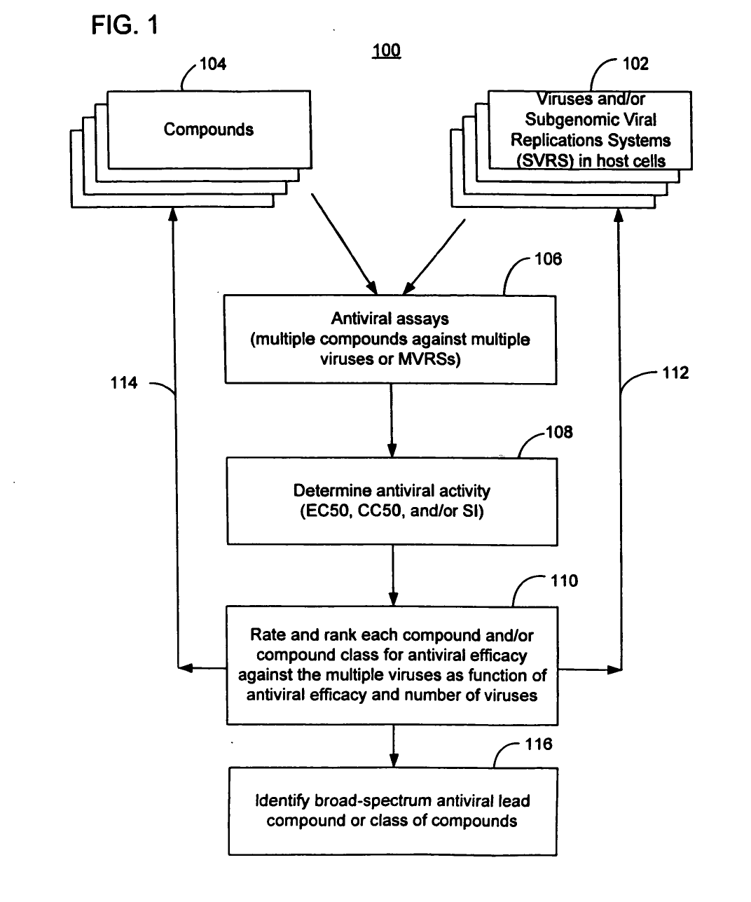

FIG. 1 illustrates another embodiment of a broad-spectrum antiviral compound screening method 100. As illustrated, two or more viruses and/or Subgenomic Viral Replication Systems (SVRS) representative of one or more of the two or more viruses are selected for assaying and one or more host cells each or group of each are prepared for assaying. A library or set of compounds 104 to be screened are selected from a total available library of compounds. One or more antiviral assays 106 are performed for each of the compounds 104 against the two or more virus/host cell 102 combinations. The antiviral assays 106 may be multiple assays of each compound 104 against multiple wells each containing a single virus/host combination 102, or may be each compound 104 against a single well containing multiple virus/host combinations 102. The antiviral assays 106 provide a determination of a data quantification of antiviral activity of each compound 104 against each virus/host combination 102. These may include one or more of an EC50, CC50, TI, and quantitative structure-activity relationship (QSAR).

Each of the quantified antiviral data are reviewed, and each are rated in block 110 for broad-spectrum antiviral efficacy as a function of antiviral activity and as a function of the number of viruses for which each compound demonstrated antiviral activity. As one embodiment, the ranking and rating of compounds is a function of each compound demonstrating antiviral activity greater than a predetermined threshold level. After rating, all compounds are ranked relative to each other based on the broad-spectrum antiviral efficacy of each compounds or class of compounds. Once each compound or class of compounds is rated and ranked as in block 110, method 100 provides that broad-spectrum antiviral lead compounds or classes of compounds may be identified in block 116.

In the alternative, in some embodiments, further assaying and screening may be performed on those compounds or classes based on their rating and ranking in block 110. For example, after block 110 additional compounds or compounds within identified classes of compounds may be identified for screening and assaying and as such method 100 provides for a feedback loop 114 for further compound selection 104. In the alternative, feedback loop 112 provides for further selection or identification of viruses, Subgenomic Viral Replication Systems (SVRS), and or host cells for further assaying. In this case, as explained elsewhere, this may include the identification and selection of different viruses within the same family or genus, or may be viruses from different families, genus's, or subgenus's. Additionally, this may include the selection of a live virus in lieu of a Subgenomic Viral Replication Systems (SVRS) or may be a Subgenomic Viral Replication Systems (SVRS) representing a different virus such as a virus having a higher hazardous or bio-safety rating. The method identified in block 102 to 110 may be performed a single time or two or more times so as to refine the rating and ranking of compounds in block 110 and thereby provide for the identification of broad-spectrum lead compounds or classes of compounds in block 116.

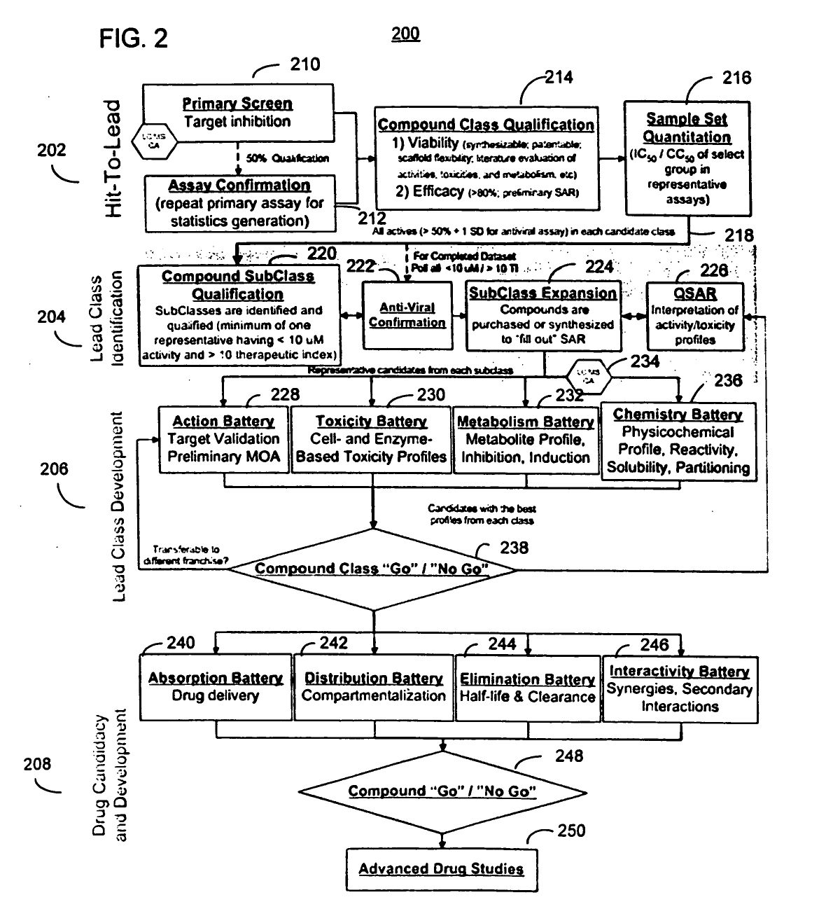

FIG. 2 illustrates yet another embodiment of a broad-spectrum antiviral compound screening and lead broad-spectrum antiviral compound selection method 200 which includes identifying broad-spectrum drug candidacy. As illustrated, four groups of method activities include a hit-to-lead block 202, a lead class identification block 204, a lead class development block 206, and a drug candidacy and development block 208.

In the hit-to-lead block 202, a primary assay or screen 210 is performed as discussed elsewhere with the library of compounds being screened against two or more viruses and/or one or more Subgenomic Viral Replication Systems (SVRS) representing one or more of the viruses. Additionally, one or more host cells may be used as the host for the two or more viruses and one or more or SVRS's. The primary screen 210 may be used to identify a target inhibition but this is not required, only that replication is inhibited as desired or established by a viral replication threshold such as greater than 50 percent inhibition such as cell cytotoxicity of CC50 greater than 50 percent. Compounds that exhibit greater than the threshold antiviral efficacy may also be confirmed by an optional assay confirmation block 212. In the assay confirmation 212, the primary assay 210 is repeated to generate confirmation antiviral efficacy statistics. Next, a broad-spectrum antiviral compound class qualification block 214 qualifies each compound class exhibiting potential antiviral efficacy. These may include all compound classes having one or more compounds having greater than an 80 percent antiviral efficacy. However, other thresholds are also possible. In block 214, the viability of the potential compound classes is reviewed to determine the ability to synthesize the compounds within the class, the potential novelty of the compound class, the scaffold flexibility, and the identification of known toxicities, activities against targets, and metabolic activities. These may be done by research such as researching technical material, databases, or literature.

A preliminary structure-activity relationship (SAR) is performed for each class of potential broad-spectrum antiviral compounds. Each potential compound class is analyzed to prepare a sample set quantification 216. In this block, the IC50 and CC50 are quantified and a therapeutic index is determined. From these, the set of potential broad-spectrum antiviral compound classes is narrowed for further study. This may be performed by a rating and ranking process as described elsewhere or may be a selection of all compound classes having antiviral efficacy greater than a particular predetermined level or percentage. For example, in one embodiment each class of compounds with all compounds within the class having greater than 50 percent efficacy within one standard deviation are selected for further broad-spectrum screening in the lead class identification block 204.

In block 204, compound subclasses are identified and qualified in block 220. A compound subclasses may be qualified when the subclass has one or more compound members with an effective concentration EC50 of less than 10 uM and a Selectivity Index (SI) greater than 10. Additionally, a complete dataset poll of all potential broad-spectrum antiviral compound classes may be performed for all compounds having an EC50 of less than 10 uM and a SI greater than 10. An antiviral confirmation assay in block 222 is performed to verify and confirm the prior assay results and quantification to ensure that only those compounds and compound classes are included in further screening and broad-spectrum antiviral lead development. For those compounds and subclasses of compounds that are confirmed in block 222, a subclass expansion block 224 is performed. In this block, the compounds within the subclasses are expanded by adding additional compounds either from an exiting library, from new or additional compound libraries, or by being synthesized. The expansion of the compounds with the subclass provided for an expanded structure-activity relationship (SAR) evaluation. From this, a quantitative structure-activity relationship (QSAR) is determined in block 226 in order to interpret the toxicity or antiviral activity profiles of each subclass of compounds. Additionally, after the subclass has been expanded in block 224, the lead class development block 206 is performed on each expanded subclass of compounds. Each of the functions performed within block 206 may be performed in series or in parallel, the later being illustrated with will provide for reduced time required for block 206.

The first sub-block is the action battery 228 provides target validation preliminary MOA is performed. In this sub-block, a target which may be commonly inhibited by the compounds within the subclass is preliminarily identified where possible. In sub-block 230, a toxicity battery is performed to develop both cell-based and enzyme-based toxicity profiles for each compound within each subclass of compounds. In sub-block 232, a metabolism battery is performed which includes determining a metabolite profile for inhibition and induction of each compound in each subclass of compounds. To ensure quality of the processes, in block 234 a LC/MS quality assurance block is performed for each compound within each subclass of compounds and prior to a chemistry battery that is performed in block 236. The chemistry battery 236 provides a physicochemical profile for each compound within each subclass of compounds, and reactivity, solubility and partitioning of each compound subclass determined. After an initial set of lead class development 206 batteries of 228, 230, 232, 234, and 236 are performed, the candidates from each class with the best profiles for broad-spectrum antiviral drug development are reviewed based on the results of the full set of lead class development batteries 228, 230, 232, 234, and 236 and the lead class compounds for drug development are selected. This selection process is a function of the objective evaluation of the results of each of these batteries and optionally based on the identification of other objective and subjective criteria. These may include identification of uniqueness and novelty of the lead class for drug development, the projected costs, the set of viruses for which each compound class demonstrated broad-spectrum antiviral efficacy, the projection potential revenues, and/or the clinical and medical benefits expected from a broad-spectrum drug developed from each compound. In some cases, a class of compounds may be transferred to a different franchise or may be identified for virus-specific drug development rather than broad-spectrum antiviral drug development.

Those compounds of classes of compounds that are selected for broad-spectrum antiviral drug development are then input into the fourth block, the drug candidacy and development block 208. As with block 206, block 208 includes several batteries of tests and analysis that may be performed in series or in parallel. These include an absorption battery 240, a distribution battery 242, an elimination battery 244, and an interactivity battery 246. In the absorption battery 240, the delivery of each proposed broad-spectrum antiviral compound into the proposed treatment host, such as a human, is tested and analyzed. The distribution battery 242 tests and determines the compartmentalization of each compound. The elimination battery 244 tests and determines the half-life and clearance of the compound in the treatment host. The interactivity battery 246 tests and identifies synergies associated with the compound and secondary interactions with other compounds or drugs.

After the drug candidacy and development sub-block batteries 240, 242, 244, and 246 are complete, each compounds and class of compounds is again evaluated for selection of a drug candidacy compound or compound class in block 248. The selection is based on objective evaluation of the results of batteries 240, 242, 244, and 246 along with previously prepared quantitative and qualitative data and analysis. A selected compound for broad-spectrum antiviral activity of block 248 is thereafter prepared for advanced drug studies in block 250 to further the development of a broad-spectrum antiviral drug from the one or more selected broad-spectrum antiviral compounds or classes of compounds. The advanced drug studies 250 may include further tests and clinical studies as required for regulatory or other purposes. The blocks and sub-blocks identified in FIG. 2 are illustrative of one embodiment. One or more blocks or sub-blocks may be optional. Additionally, one or more additional blocks or sub-blocks may be added or repeated.

Discovery of Viral Replication Targets

The broad-spectrum method described herein also provides a new method for finding an unknown target associated with viral replication. Such an unknown target may be a protein that is required for the replication of multiple viruses. By using the broad-spectrum screening method, a target may be identified as a function of efficacy of one or more compounds against two or more viruses through analysis of the quantifiable screening data and a rating system. For example, in one embodiment assays of a compound against multiple viruses and one or more hosts will result in nearly identical measurement and/or antiviral ratings. In such cases, an equivalent rating is one indication of the presence of a target which, when addressed by the compound, produces a common antiviral efficacy against the common protein or target present in each of the viruses or required for replication by each of the viruses.

Selection of such a compound and analysis of the broad-spectrum data identifies new target proteins for additional analysis and research. Such a new target protein may then be further studied and screened either in the broad-spectrum screening method or in a target-based screening method. As such, while the broad-spectrum method is not a target-based screening process, the broad-spectrum method and associated quantified screening data provides for identification of targets, which may subsequently be used for further target-based screening with the lead compound identified during the broad-screening method or with other compounds that have potentially high efficacies against the identified target.

Additionally, a broad-spectrum antiviral compound may have a SAR/QSAR that tracks together or has similar quantitative measurements for two or more viruses. When broad-spectrum antiviral quantitative measurements are proportionally or are common among two or more viruses, this is indicative of a potentially common target, even though the target itself is unknown.

This is in contrast to the other drug discovery and screening methods where the screening and medical chemistry process focuses on compound that produces increases in QSAR or other quantifiable measurements for a known and desired target, but decreasing for others, e.g., in other drug discovery methods, screening prefers the antiviral efficacy measurements such as QSAR diverge thereby indicating a selectivity towards the particular target or virus. In target-based or virus-specific screening, an antiviral efficacy measurement that increases for one virus and decreases for a second virus is desired as this may be indicative of antiviral efficacy against the particular virus or target.

Drug Development and Marketing of a Broad-Spectrum Drug

The discovery of compounds with broad-spectrum antiviral efficacy consistent with the broad-spectrum antiviral compound screening and discovery method provides improvements to existing methods of antiviral drug development, regulatory approval of the antiviral drugs, the marketing and distribution of antiviral drugs and the treatment of virus infections in patients by medical service providers. The broad-spectrum discovery company may sell, provide a license, or otherwise transfer the broad-spectrum antiviral compound to the drug company to produce and market a broad-spectrum antiviral drug from the broad-spectrum antiviral compound. Such arrangements typically provide for the payment of fees, milestone payments, and/or royalty payments to the drug discovery company as a function of the drug company's marketing and sale of any associated broad-spectrum or target-specific drug developed from the broad-spectrum antiviral compound.

In one embodiment, a method includes the delivery of a broad-spectrum antiviral compound to a drug company by a drug discovery company. As discussed above, a broad-spectrum antiviral compound having antiviral activity against two or more viruses is identified by an antiviral compound screening and/or discovery company. The antiviral screening company provides information obtained during the broad-spectrum screening method to the drug company about the broad-spectrum antiviral compound. This will include identification of the two or more viruses for which the broad-spectrum compound has demonstrated antiviral activity. Additionally, additional information is provided with regard to any potential classes of compounds and their potency against two or more viruses. The information may also include the identification of the treatment of antiviral infection against two or more viruses each of which individually is considered a unique and separate market opportunity for an antiviral drug. In such cases, market opportunities may be aggregated due to the broad-spectrum antiviral efficacy of the compound and associated drugs.

As a potential broad-spectrum antiviral drug, an aggregate of potential market opportunities for the broad-spectrum antiviral drug may be considered by the drug company. Each of the individual market opportunities, may themselves, be considered to be unprofitable for antiviral drug development. While there are large market opportunities related to chronic, high prevalence and/or high incidence viruses such as Human immunodeficiency virus (HIV), Hepatitis C virus (HCV) and the Herpes viruses (HSV, VZV, EBV, CMV, etc.), and mid-sized markets that include acute and/or high incidence viruses such as Influenza A and B, and respiratory syncytial virus (RSV), viruses that have low prevalence and low incidence may not be attractive markets for virus-specific drug development. These may include respiratory viruses such as SARS, PIV1-3, hMPV, and rhinoviruses; Enteric viruses such as rotavirus, enterovirueses, and caliciviruses; Encephalitis viruses such as VEE, JE, and TBE; Hepatitis viruses such as hepatitis A, B and E; and Hemorrhagic fever viruses such as Ebola, Marburg, and Lassa fever. In these cases, an opportunity to aggregate one or more viruses through the development of a broad-spectrum antiviral drug may create new opportunities for a drug company and therefore for treatment. A compound with broad-spectrum antiviral activity enables a drug company to aggregate these smaller market opportunities either together or in conjunction with one or more large or mid-sized markets. By aggregating smaller and/or potentially unprofitable market opportunities, the drug company is able to justify the considerable expense required to develop a broad-spectrum drug, to obtain regulatory approval, and to market and distribute to medical care service providers.

Additionally, one of the aggregate of market opportunities may include increasing an antiviral efficacy of a particular or virus-specific antiviral drug against a particular virus. For instance, a particular virus-specific drug may have known antiviral efficacy against HCV. However, it may be desired by the drug company or by a medical care provider to increase the antiviral efficacy of the virus-specific drug. In such a case, a broad-spectrum antiviral drug may be used in combination with the virus-specific drug to increase the antiviral efficacy against HCV or to provide enhanced antiviral efficacy in the patient.

In another embodiment, one of the aggregate of market opportunities may be the treatment of a suspected viral infection prior to the diagnoses of the particular virus responsible for the suspected viral infection. Another of the market opportunities that may be aggregated is the treatment of a patient for a particular viral infection associated with a particular virus where and when a particular antiviral drug is unavailable. Unavailability may be due to the lack of a known antiviral drug for the particular virus, or it may be due to a current shortage or out of stock condition of the particular antiviral drug at the location of the health care provider or the patient.

In the development of a broad-spectrum antiviral drug from a broad-spectrum antiviral compound, the drug company may perform one or more clinical trials associated with the treatment of two or more viral infections associated with the two or more viruses. Such a clinical trial may include the trial of the broad-spectrum drug in the treatment of a suspected viral infection prior to a diagnosis of the virus responsible for the viral infection. This may include early treatment prior to diagnosis or may be treatment of the patient for an unknown virus. The trial may also include treatment of a viral infection where a known virus-specific antiviral drug in unavailable or where the virus-specific antiviral drug is ineffective. In such cases, this may a trial of the treatment with one or more combinations of antiviral drug in combination with the broad-spectrum antiviral drug.

These methods also apply to the marketing of a broad-spectrum antiviral compound to a health care provider for treatment of a patient having a virus infection. In this embodiment, after a broad-spectrum antiviral compound is identified and the information about the broad-spectrum antiviral activity is provided to the drug company, the drug company may provide information related to the broad-spectrum antiviral efficacy against the two or more viruses to one or more health care providers. This information may be in the form of advertisement, a webpage, emails, direct mailing, seminars, literature, and/product inserts. A health care provider may place an order with the drug company for a broad-spectrum drug containing the broad-spectrum antiviral compound and the drug company delivers the broad-spectrum drug to the health care provider or to the patient of the healthcare provider in response to the placement of an order or a request. This may also include the provision of samples by the drug company to the health care provider and the delivery of those samples to a patient by the health care provider. Additionally, in response to receiving a request for delivery from the health care provider, the drug company may receive a payment.

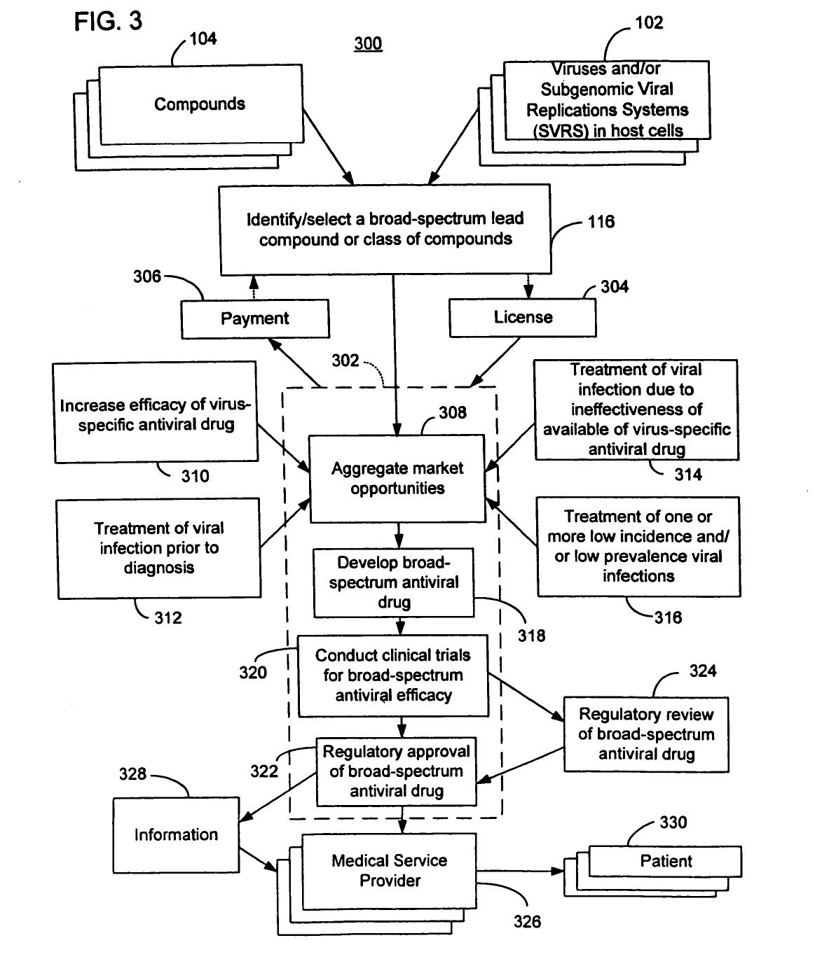

FIG. 3 illustrates one embodiment of a method 300 for developing and delivering a broad-spectrum antiviral drug for patient treatment. As discussed above, multiple compounds 104 are assayed and screened for broad-spectrum antiviral efficacy against two or more viruses which may include one or more Subgenomic Viral Replication Systems (SVRS) contained in one or more host cells 102. One or more compounds or classes of compounds are identified and selected in block 116. As illustrated, blocks 102, 104, and 116 may be performed by an entity performing these broad-spectrum antiviral compound screening blocks. In such a case, once the broad-spectrum antiviral compound is identified, the screening entity may provide a license 304 to a drug development entity 302 for the development of one or more broad-spectrum or a virus-specific drugs. As a function of the license 304, drug development entity 302 makes one or more payments to the screening entity. Of course, in some embodiments, the broad-spectrum screening entity and the drug development entity may be a single entity and the license 304 and payment 306 may be an internal entity function or transfer or arrangement.

Drug development entity 302 would obtain the license of block 304 as a function of the ability to aggregate in block 308 two or more market opportunities which are associated with the broad-spectrum antiviral efficacy of the broad-spectrum compound. As an example, the aggregation of market opportunities 308 may include two or more market opportunities which may include increasing the antiviral efficacy of a virus-specific antiviral drug 310, treatment of a viral infection in a patient prior to diagnosis of the virus responsible for the viral infection 312, treatment of a viral infection in a patient where an virus-specific antiviral drug is either ineffective against the virus or is unavailable for treatment of the patient 314, and treatment of one or more low incidence or low prevalence viral infections 316. In the later case, one or more low incidence or low prevalence viral infections, may be such that each individually due not warrant or financially justify the financial expenditures required to bring a drug to market. In such a case, drug development entity 302 would aggregate the revenue opportunities associated with each low incidence and low prevalence viral infections due to the broad-spectrum antiviral efficacy of the compound or class of compounds. One or more of these market opportunities may be aggregated thereby enabling drug development entity 302 in block 308 to justify the license 304 of the broad-spectrum compound and the associated payment 306.

After obtaining license in block 304 as a function of aggregated market opportunities of block 308, drug development entity 302 develops one or more broad-spectrum antiviral drugs in block 318. This may include the conducting of one or more clinical trials of block 320 to support broad-spectrum antiviral drug efficacy and use as a drug for the intended purpose in the intended treatment host. These clinical trials 320 may be required for regulatory review in block 324 and approval of a broad-spectrum antiviral drug in block 322 by a regulatory entity. In association with one or more of the trials and in association with regulatory approval of block 322, information or data related to the broad-spectrum antiviral efficacy of the compounds and drugs is provided in block 328 to one or more medical service providers 326. Information 328 may be in the form of seminars, trade show presentations, website, email, mailings, package inserts, sales visits, etc, which contain data and information about the antiviral efficacy of the broad-spectrum drug against two or more viruses or two or more viral infections. Medical service provider 326 provides information to one or more patients 330 who may require treatment of one or more of the viral infections or viruses for which the broad-spectrum antiviral drug may have antiviral activity. This may be in the form of a prescription to the patient of the broad-spectrum antiviral drug.

Of course, one or more of these activities may be provided by a single person or company or a combination of persons, companies or entities consistent with the described method.

Treatment of Viral Infections with Broad-Spectrum Antiviral Drugs

One or more embodiments of the broad-spectrum antiviral method described herein provide a medical care provider with new and improved methods of therapy and treatment against viral infections. In one embodiment of the present invention, a suspected viral infection is treated by a medical provider as an initial stage therapy prior to determination of the presence of a viral infection or it may be prior to the identification of a particular type or strain of virus. By providing a patient suspected of having a viral infection with a broad-spectrum antiviral compound or drug, the patient is provided with an early-stage treatment which will inhibit rapid replication of the virus within the patient thereby providing the medical care providers with increased time to diagnose the patient's condition and determine the virus type producing the viral infection and the application of any virus-specific treatment.

Additionally, a broad-spectrum antiviral drug may be administered to a patient as a therapy against an unknown virus, such as prior to the identification and classification of the SARS virus in 2002. In the example of SARS, a broad-spectrum antiviral compound may have been administered to a patient suspected of having a virus, even though the existence of or type of virus causing the viral infection was unknown. Again, in such a treatment, administration of the broad-spectrum antiviral compound at an early stage provides for antiviral treatment which may inhibit replication of any virus within the patient.

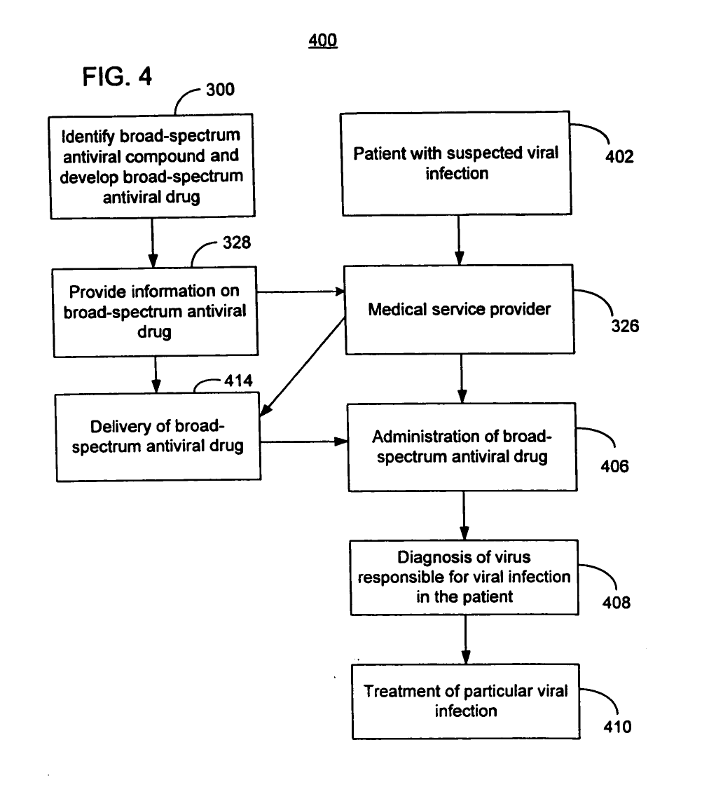

Referring now to FIG. 4, a method of treatment of a patient with a suspected viral infection is illustrated as 400. As discussed above, a broad-spectrum antiviral compound is identified and a broad-spectrum antiviral drug is developed as in block 300. After the broad-spectrum antiviral drug is developed, information is developed and provided to the medical service provider 326 as shown in block 328.

In block 402, a patient has medical condition that may be a viral infection. The patient receives medical services and diagnosis from a medical service provider 326 such as a doctor, nurse, nurse practitioner, etc. The medical service provider having diagnosed the patient and having reviewed the provided information on the broad-spectrum antiviral drug, provides a request for delivery of a broad-spectrum antiviral drug as in block 414 as a function of the information provided in block 328. The broad-spectrum antiviral drug is delivered to the medical service provider or patient for administration of the broad-spectrum antiviral drug to the patient as in block 406. The administration of the broad-spectrum antiviral drug in block 406 is prior to the diagnosis or determination of a viral infection responsible for the patient's condition or prior to the diagnosis of the particular viral infection and/or virus.

The delivered broad-spectrum antiviral drug of 414 may be administered in block 406 as a precautionary measure or as form of an immediate medication due to the patient's condition or due to a suspected virus from the symptoms. The virus may be one is later diagnosed from known viruses or may be an unknown virus, such as during the early period of the SARS outbreak. After administration of the broad-spectrum antiviral drug to the patient in block 406, the virus responsible for the viral infection in the patient is diagnosed in block 408. In this embodiment, in block 410, once the particular virus associated with the viral infection is known, the patient is treated with a drug or medical procedure for the particular viral infection. This may include continuing to administer the broad-spectrum antiviral drug alone or in combination with one or more other drugs that have known antiviral efficacy against the diagnosed virus.

In another embodiment, a broad-spectrum antiviral drug may be administered to a patient as a therapy against a known virus where there is no known virus-specific antiviral drug known at the time or where the virus-specific antiviral drug does not have effective efficacy to control or inhibit the virus. For example, after the SARS virus was identified and therefore diagnosable in a patient, there may be no known antiviral drug available for treatment of the patient. In such a case, one or more broad-spectrum antiviral drugs may be administered to the patient to suppress, delay, or treat the known virus to the extent capable of the broad-spectrum antiviral drug.

In another embodiment, a broad-spectrum antiviral drug may also be provided to a patient as a treatment in conjunction with one or more other drugs which may have known antiviral activity against an identified virus. In this embodiment, the broad-spectrum antiviral is provided in a “cocktail” therapy which may provide separate additionally antiviral activity against the known virus or may act as a catalyst to improve the efficacy of one or more other antiviral drugs. For example, a broad-spectrum may be administered in combination with another antiviral drug such as interferon-α to treat hepatitis C virus.

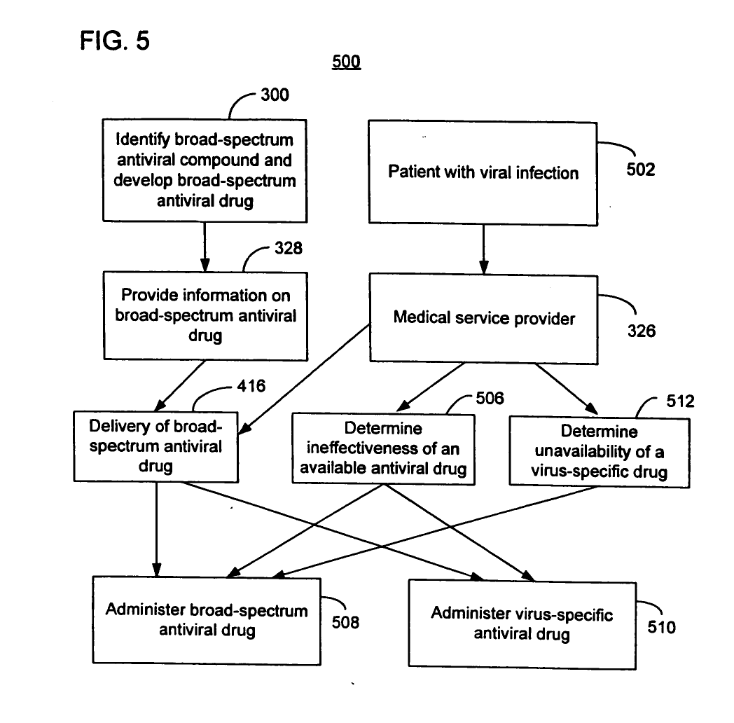

Referring to FIG. 5, a method of treatment of a patient with a viral infection is illustrated as 500. As discussed above, a broad-spectrum antiviral compound is identified and a broad-spectrum antiviral drug is developed as in block 300. After the broad-spectrum antiviral drug is developed, information is developed and provided to the medical service provider 326 as shown in block 328.

As shown, a patient has a viral infection in block 502 and receives medical services from a medical service provider 326. The medical service provider 326 provides a request for delivery of a broad-spectrum antiviral drug as in block 414 as a function of the provided information on the broad-spectrum antiviral drug of block 328.

The medical service provider may identify the particular virus responsible for the viral infection in the patient and identify that one or more available antiviral drugs for the particular virus are ineffective as in block 506. In this case, a broad-spectrum antiviral drug may be administered to the patient in lieu of the particular virus-specific antiviral drug of block 508 and as an option may be administered in conjunction with administration of the virus-specific antiviral drug as in block 510.

In the alternative, the medical service provider 326 may determine that a virus-specific drug is not available for the particular virus as in block 512. This may be due to limited supplies of a known virus-specific drug or may be due to a virus-specific antiviral drug not being known. In either case, the broad-spectrum antiviral drug is requested for delivery to the medical service provider 326 or to the patient as in block 414. The broad-spectrum antiviral drug is administered to the patient in block 508 for treatment of the viral infection.

When introducing aspects of the invention or embodiments thereof, the articles “a”, “an”, “the”, and “said” are intended to mean that there are one or more of the elements. The terms “comprising”, “including”, and “having” are intended to be inclusive and mean that there may be additional elements other than the listed elements.

It is further to be understood that the steps described herein are not to be construed as necessarily requiring their performance in the particular order discussed or illustrated unless specifically stated as such. It is also to be understood that additional or alternative steps may be employed.

The above description is merely exemplary in nature and is not intended to limit the invention, its application, or uses. In view of the above, it will be seen that several aspects of the invention are achieved and other advantageous results attained. As various changes could be made in the above exemplary constructions and methods without departing from the scope of the invention, it is intended that all matter contained in the above description or shown in the accompanying drawings shall be interpreted as illustrative and not in a limiting sense. Further aspects of the present invention are apparent from the detailed description provided hereinafter. The detailed description and specific examples, while indicating one or more embodiments of the invention, are intended for purposes of illustration only and are not intended to limit the scope of the invention.

1. A method for identifying a broad-spectrum antiviral lead compound, the method comprising:
determining antiviral activity of a plurality of compounds against two or more viruses; and
identifying a broad-spectrum antiviral lead compound from the plurality of compounds, said lead compound having activity against at least two of the two or more viruses.

2. The method of claim 1 wherein determining comprises determining antiviral activity of each of the plurality of compounds against at least one subgenomic viral replication system representative of at least one of the two or more viruses.

3. The method of claim 2 wherein the subgenomic viral replication system is selected from a group consisting of a defective genome, a minigenome, an amplicon, and a replicon.

4. The method of claim 1 wherein identifying is a function of the determined antiviral activity and a number of the two or more viruses for which the lead compound has antiviral activity.

5. The method of claim 1 wherein the determining of antiviral activity for each of the plurality of compounds comprises determining one or more of the group consisting of an EC50, a CC50, and a Selectivity Index (SI).

6. The method of claim 1 wherein identifying a broad-spectrum antiviral lead compound is a function of rating each of the plurality of compounds for broad-spectrum antiviral activity, said rating being a function of the number of viruses for which each compound has antiviral activity.

7. The method of claim 1 wherein the identified broad-spectrum antiviral lead compound has antiviral activity greater than a predetermined threshold antiviral activity against each of the at least two of the two or more viruses.

8. The method of claim 1 wherein the two or more viruses comprise two or more viruses from one viral family and the identified broad-spectrum antiviral lead compound has antiviral activity greater than a predetermined threshold antiviral activity against at least two viruses of the two or more viruses from the one viral family.

9. The method of claim 1 wherein the two or more viruses are RNA viruses.

10. The method of claim 1 wherein the two or more viruses are positive-strand RNA viruses.

11. The method of claim 1 wherein the two or more viruses are from one or more virus families selected from a group consisting of Picornaviridae, Caliciviridae, Astroviridae, Coronaviridae, Togaviridae, and Flaviviridae.

12. The method of claim 1 wherein the two or more viruses are selected from a group consisting of Sindbis virus, rubella, hepatitis C virus, West Nile virus, yellow fever virus, tick-borne encephalitis virus, Japanese encephalitis virus, coxsackivirus, enterovirus, hepatitis A virus, SARS, astrovirus, Dengue fever virus, poliovirus, and Venezuela encephalitis virus, WEE, EEE, Marayo O'nong nong, Ross River, Chikungunya, DV, Rhinovirus, Feline, murine, Norwalk, Bovine, and human coronaviridae.

13. The method of claim 1 wherein the two or more viruses are negative-strand RNA viruses.

14. The method of claim 1 wherein the two or more viruses are from one or more virus families selected from a group consisting of Paramyxoviridae, Rhabdoviridae, Filoviridae, Orthomyxoviridae, Bunyaviridae, Bornaviridae, and Arenaviridae

15. The method of claim 1 wherein the two or more negative-strand RNA viruses consist of RSV, Ebola, and Influenza.

16. The method of claim 1 wherein the two or more viruses are selected from a group consisting of respiratory syncytial virus (RSV), Ebola virus, rabies virus, Lassa fever, Argentine hemorrhagic fever virus, La Crosse virus, Rift Valley fever, Hantaan virus, California encephalitis virus, influenza virus A, influenza virus B, measles, mumps, Marburg virus, Bolivian hemorrhagic fever virus, Crimean-Congo virus, HPIV, HMPV, Nipah, Hendra, VSV, LCMV, Junin, Bunyamwera, Uukuniemi, and CCHF.

17. The method of claim 1 wherein the two or more viruses are RNA retroviruses.

18. The method of claim 1 wherein the two or more viruses are selected from a group consisting of HIV-1, HIV-2, HTLV-1, and HTLV-2.

19. The method of claim 1 wherein the two or more viruses are double strand RNA viruses.

20. The method of claim 1 wherein the two or more viruses includes a virus from a Reoviridae virus family.

21. The method of claim 1 wherein the two or more viruses are DNA viruses.

22. The method of claim 1 wherein the two or more viruses are from one or more virus families selected from a group consisting of Herpesviridae, Polyomaviridae, Papillomavirdidae, Adenoviridae, and Parvovirdae.

23. The method of claim 1 wherein the two or more viruses are selected from a group consisting of human parvovirus, adeno-associated virus, herpes simplex virus type 1, herpes simplex virus type two, human herpes simplex virus type six, human herpes virus type seven, human herpes virus type eight, human adenovirus, BK virus, human papilloma virus, Epstein-Barr virus, JC virus, human cytomegalovirus, and varicella-zoster virus.

24. The method of claim 1 wherein the two or more viruses are DNA reverse transcribing viruses.

25. The method of claim 1 wherein the two or more viruses includes a virus from Hepadnaviridae family.

26. The method of claim 1 wherein the two or more viruses are selected from a group consisting of RNA viruses and DNA viruses.

27. The method of claim 1 wherein the two or more viruses are selected from a group consisting of hepatitis C virus, yellow fever virus, respiratory syncytial virus, Sindbis virus, influenza virus A, Venezuela encephalitis virus, West Nile virus, and Ebola virus.

28. The method of claim 1 wherein the two or more viruses are selected from a group consisting of respiratory syncytial virus and hepatitis C virus.

29. The method of claim 1 wherein the two or more viruses are selected from a group consisting of West Nile virus, yellow fever virus, Sindbis virus, Venezuela encephalitis virus, and Ebola virus.

30. The method of claim 1 wherein the two or more viruses are from one or more virus families selected from a group consisting of Herpesviridae, Polyomaviridae, Papillomavirdidae, Adenoviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Bornaviridae, Picornaviridae, Caliciviridae, Astroviridae, Coronaviridae, Togaviridae, and Flaviviridae.

31. The method of claim 1 wherein the two or more viruses are selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

32. A method for identifying a class of broad-spectrum antiviral compounds, the method comprising:
determining antiviral activity of compounds from two or more classes of compounds against two or more viruses, each of said classes of compounds having one or more member compounds; and
identifying a class of broad-spectrum antiviral compounds, said class of broad-spectrum antiviral compounds having a member compound with antiviral activity greater than a predetermined threshold antiviral activity against a plurality of the two or more viruses.

33. The method of claim 32 wherein determining comprises determining antiviral activity of each compound against at least one subgenomic viral replication system representative of at least one of the two or more viruses.

34. The method of claim 33 wherein the subgenomic viral replication system is selected from a group consisting of a defective genome, a minigenome, an amplicon, and a replicon.

35. The method of claim 32 wherein the two or more viruses comprise two or more viruses from one viral family and the identified class of broad-spectrum antiviral compounds has activity greater than the predetermined threshold antiviral activity against at least two viruses of the two or more viruses from the one viral family.

36. The method of claim 32 wherein the two or more viruses are RNA viruses.

37. The method of claim 32 wherein the two or more viruses are DNA viruses.

38. The method of claim 32 wherein the two or more viruses are selected from a group consisting of positive-strand RNA viruses, negative-strand RNA viruses, RNA reverse transcribing viruses, double strand RNA viruses, and DNA viruses.

39. The method of claim 32 wherein the two or more viruses are from one or more virus families selected from a group consisting of Herpesviridae, Polyomaviridae, Papillomavirdidae, Adenoviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Picornaviridae, Caliciviridae, Astroviridae, Coronaviridae, Togaviridae, and Flaviviridae.

40. The method of claim 32 wherein the two or more viruses are selected from a group consisting of respiratory syncytial virus and hepatitis C virus.

41. The method of claim 32 wherein the two or more viruses are selected from a group consisting of West Nile virus, yellow fever virus, Sindbis virus, Venezuela encephalitis virus, and Ebola virus.

42. The method of claim 32 wherein the two or more viruses are selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

43. A method of rating compounds for broad-spectrum antiviral efficacy, the method comprising:
determining antiviral activity for compounds against two or more viruses; and
rating each compound for broad-spectrum activity as a function of the determined antiviral activity and a number of viruses for which each compound has antiviral activity.

44. The method of claim 43 wherein the two or more viruses includes at least one subgenomic viral replication system representative of at least one of the two or more viruses.

45. The method of claim 44 wherein the subgenomic viral replication system is selected from a group consisting of a defective genome, a minigenome, an amplicon, and a replicon.

46. The method of claim 43 wherein the determining of antiviral activity for compounds and the rating of each compound comprises determining and rating as a function of one or more antiviral measurements from the grouping consisting of an EC50, a CC50, and a Selectivity Index (SI).

47. The method of claim 43 wherein the rating of each compound for broad-spectrum antiviral activity is proportional to the number of viruses for which each compound has antiviral activity.

48. The method of claim 43 wherein the rating of each compound for broad-spectrum antiviral activity is a function of a number of viruses for which each compound has antiviral activity greater than a predetermined threshold antiviral activity rating level.

49. The method of claim 48, further comprising selecting a broad-spectrum lead compound as a function of the rating of the compounds.

50. The method of claim 48 wherein selection a broad-spectrum lead compound is a function of a rating greater than a predetermined threshold antiviral activity rating level.

51. The method of claim 43 wherein the two or more viruses are RNA viruses.

52. The method of claim 43 wherein the two or more viruses are DNA viruses.

53. The method of claim 43 wherein the two or more viruses are selected from a group consisting of positive-strand RNA viruses, negative-strand RNA viruses, RNA reverse transcribing viruses, double strand RNA viruses, and DNA viruses.

54. The method of claim 43 wherein the two or more viruses are from one or more virus families selected from a group consisting of Herpesviridae, Polyomaviridae, Papillomavirdidae, Adenoviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Bornaviridae, Picornaviridae, Caliciviridae, Astroviridae, Coronaviridae, Togaviridae, and Flaviviridae.

55. The method of claim 43 wherein the two or more viruses are selected from a group consisting of respiratory syncytial virus and hepatitis C virus.

56. The method of claim 43 wherein the two or more viruses are selected from a group consisting of West Nile virus, yellow fever virus, Sindbis virus, Venezuela encephalitis virus, and Ebola virus.

57. The method of claim 43 wherein the two or more viruses are selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

58. A method for developing and marketing a broad-spectrum antiviral lead compound, the method comprising:
selecting a broad-spectrum antiviral lead compound;
developing a broad-spectrum antiviral drug from the broad-spectrum antiviral lead compound; and
marketing the broad-spectrum antiviral drug to an aggregate of market opportunities, said aggregate of market opportunities includes treatment of two or more viral infections associated with two or more viruses.

59. The method of claim 58 wherein one of the aggregate of market opportunities comprises increasing an antiviral efficacy of a particular antiviral drug against a particular virus.

60. The method of claim 58 wherein one of the aggregate of market opportunities is treatment of a suspected viral infection prior to diagnosing a particular virus responsible for the suspected viral infection.

61. The method of claim 58 wherein one of the aggregate of market opportunities comprises treatment of a patient for a particular viral infection associated with a particular virus as a function of the unavailability of a particular antiviral drug having antiviral efficacy against the particular virus.

62. The method of claim 58, further comprising performing a clinical trial of the broad-spectrum drug in the treatment of the two or more viral infections associated with the two or more viruses.

63. The method of claim 58, further comprising obtaining regulatory approval of the broad-spectrum drug for the treatment of the two or more viral infections associated with the two or more viruses.

64. The method of claim 58 wherein the two or more viruses are RNA viruses.

65. The method of claim 58 wherein the two or more viruses are DNA viruses.

66. The method of claim 58 wherein the two or more viruses are selected from a group consisting of positive-strand RNA viruses, negative-strand RNA viruses, RNA reverse transcribing viruses, double strand RNA viruses, and DNA reverse transcribing viruses.

67. The method of claim 58 wherein the two or more viruses are from one or more virus families selected from a group consisting of Herpesviridae, Polyomaviridae, Papillomavirdidae, Adenoviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Bornaviridae, Picornaviridae, Caliciviridae, Astroviridae, Coronaviridae, Togaviridae, and Flaviviridae.

68. The method of claim 58 wherein the two or more viruses are selected from a group consisting of respiratory syncytial virus and hepatitis C virus.

69. The method of claim 58 wherein the two or more viruses are selected from a group consisting of West Nile virus, yellow fever virus, Sindbis virus, Venezuela encephalitis virus, and Ebola virus.

70. The method of claim 58 wherein the two or more viruses are selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

71. A method for delivering a broad-spectrum antiviral compound to a drug company, the method comprising:
identifying the broad-spectrum antiviral compound having antiviral activity against two or more viruses;
providing information to the drug company about broad-spectrum antiviral compound and an aggregate of market opportunities for the broad-spectrum antiviral compound; and
transferring the broad-spectrum antiviral compound to the drug company, said drug company producing and marketing a broad-spectrum antiviral drug from the transferred broad-spectrum antiviral compound.

72. The method of claim 71 wherein transferring consists of one or more functions selected from a group consisting of selling, contracting, licensing, receiving ownership rights, and receiving payment.

73. The method of claim 71 wherein one of the aggregate of market opportunities comprises increasing an antiviral efficacy of a particular antiviral drug against a particular virus.

74. The method of claim 71 wherein one of the aggregate of market opportunities comprises treatment of a suspected viral infection prior to diagnosing a particular virus responsible for the suspected viral infection.

75. The method of claim 71 wherein one of the aggregate of market opportunities comprises treatment of a particular viral infection associated with a particular virus as a function of an unavailability of a particular antiviral drug having antiviral efficacy against the particular virus.

76. The method of claim 71 wherein two or more of the aggregate of market opportunities comprises treatment of two or more viruses selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

77. The method of claim 71 wherein the two or more of the aggregate of market opportunities comprises treatment of two or more viruses from one or more virus families selected from a group consisting of Herpesviridae, Polyomaviridae, Papillomavirdidae, Adenoviridae, Bomaviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Picornaviridae, Caliciviridae, Astroviridae, Coronaviridae, Togaviridae, and Flaviviridae.

78. The method of claim 71 wherein the two or more viruses are RNA viruses.

79. The method of claim 71 wherein the two or more viruses are DNA viruses.

80. The method of claim 71 wherein the two or more viruses are selected from a group consisting of positive-strand RNA viruses, negative-strand RNA viruses, RNA reverse transcribing viruses, double strand RNA viruses, and DNA reverse transcribing viruses.

81. The method of claim 71 wherein the two or more viruses are from one or more virus families selected from a group consisting of Herpesviridae, Polyomaviridae, Papillomavirdidae, Adenoviridae, Bornaviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Picornaviridae, Caliciviridae, Astroviridae, Coronaviridae, Togaviridae, and Flaviviridae.

82. The method of claim 71 wherein the two or more viruses are selected from a group consisting of respiratory syncytial virus and hepatitis C virus.

83. The method of claim 71 wherein the two or more viruses are selected from a group consisting of West Nile virus, yellow fever virus, Sindbis virus, Venezuela encephalitis virus, and Ebola virus.

84. The method of claim 71 wherein the two or more viruses are selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

85. A method for marketing a broad-spectrum antiviral compound to a health care provider for treatment of a patient having a virus infection, the method comprising:
identifying the broad-spectrum antiviral compound having activity against two or more viruses,
providing information about the broad-spectrum antiviral activity of said broad-spectrum antiviral compound to the health care provider; and
delivering the broad-spectrum antiviral compound in response to receiving a request for delivery from the health care provider.

86. The method of claim 85 wherein providing information includes providing information about the antiviral activity of the broad-spectrum antiviral compound against the two or more viruses.

87. The method of claim 85 wherein providing information includes providing information about an aggregate of market opportunities for the broad-spectrum antiviral compound, said aggregate of market opportunities includes the antiviral activity of the broad-spectrum antiviral compound against the two or more viruses.

88. The method of claim 87 wherein one of the aggregate of market opportunities comprises increasing an antiviral efficacy of a particular antiviral drug against a particular virus, the particular antiviral drug having antiviral efficacy against the particular virus.

89. The method of claim 87 wherein one of the aggregate of market opportunities is treatment of a suspected viral infection prior to diagnosing a particular virus responsible for the suspected viral infection.

90. The method of claim 87 wherein one of the aggregate of market opportunities comprises treatment of a particular viral infection associated with a particular virus as a function of an unavailability of a particular antiviral drug having antiviral activity against the particular virus.

91. The method of claim 85, further comprising receiving payment in exchange for the delivery of the broad-spectrum antiviral compound.

92. The method of claim 85 wherein identifying comprises identifying the antiviral activity of the plurality of compounds against at least one subgenomic viral replication system representative of at least one of the two or more viruses.

93. The method of claim 92 wherein the subgenomic viral replication system is selected from a group consisting of a defective genome, a minigenome, an amplicon, and a replicon.

94. The method of claim 85 wherein the two or more viruses are RNA viruses.

95. The method of claim 85 wherein the two or more viruses are DNA viruses.

96. The method of claim 85 wherein the two or more viruses are selected from a group consisting of positive-strand RNA viruses, negative-strand RNA viruses, RNA reverse transcribing viruses, double strand RNA viruses, and DNA reverse transcribing viruses.

97. The method of claim 85 wherein the two or more viruses are from one or more virus families selected from a group consisting of Herpesviridae, Polyomaviridae, Papillomavirdidae, Adenoviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Bomaviridae, Picornaviridae, Caliciviridae, Astroviridae, Coronaviridae, Togaviridae, and Flaviviridae.

98. The method of claim 85 wherein the two or more viruses are selected from a group consisting of respiratory syncytial virus and hepatitis C virus.

99. The method of claim 85 wherein the two or more viruses are selected from a group consisting of West Nile virus, yellow fever virus, Sindbis virus, Venezuela encephalitis virus, and Ebola virus.

100. The method of claim 85 wherein the two or more viruses are selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

101. A method for treating a suspected viral infection in a patient by administering a broad-spectrum antiviral compound to a patient, the method comprising:
determining a presence of the suspected viral infection in the patient, said viral infection being associated with a particular virus; and
administering the broad-spectrum antiviral compound to the patient, said administering being prior to determining the particular virus responsible for the suspected viral infection.

102. The method of claim 101 wherein the broad-spectrum antiviral compound has antiviral activity against two or more viruses.

103. The method of claim 102 wherein the two or more viruses are RNA viruses.

104. The method of claim 101 wherein the two or more viruses are DNA viruses.

105. The method of claim 101 wherein the two or more viruses are selected from a group consisting of positive-strand RNA viruses, negative-strand RNA viruses, RNA reverse transcribing viruses, double strand RNA viruses, and DNA reverse transcribing viruses.

106. The method of claim 101 wherein the two or more viruses are from one or more virus families selected from a group consisting of Herpesviridae, Polyomaviridae, Papillomavirdidae, Adenoviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Bomaviridae, Picornaviridae, Caliciviridae, Astroviridae, Coronaviridae, Togaviridae, and Flaviviridae.

107. The method of claim 101 wherein the two or more viruses are selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.

108. A method for treatment of a patient having a particular viral infection, the method comprising:
determining an ineffectiveness of an available antiviral drug against a particular virus associated with the particular viral infection; and
administering a broad-spectrum antiviral compound to the patient.

109. The method of claim 108, further comprising:
determining a presence of a particular viral infection in a patient, said particular viral infection resulting from the particular virus;
determining a particular antiviral drug having antiviral activity against the particular virus; and
administering to the patient the particular antiviral drug in combination with administering the broad-spectrum antiviral compound.

110. The method of claim 108 wherein determining an ineffectiveness of an available antiviral drug is determining that a particular antiviral drug having antiviral activity against the particular virus is not available.

111. The method of claim 108 wherein the broad-spectrum antiviral compound has antiviral activity against two or more viruses.

112. The method of claim 108, further comprising identifying the broad-spectrum antiviral compound as a function of antiviral activity against two or more viruses.

113. The method of claim 108 wherein the two or more viruses are RNA viruses.

114. The method of claim 108 wherein the two or more viruses are DNA viruses.

115. The method of claim 108 wherein the two or more viruses are selected from a group consisting of positive-strand RNA viruses, negative-strand RNA viruses, RNA reverse transcribing viruses, double strand RNA viruses, and DNA reverse transcribing viruses.

116. The method of claim 108 wherein the two or more viruses are from one or more virus families selected from a group consisting of Herpesviridae, Polyomaviridae, Papillomavirdidae, Adenoviridae, Parvovirdae, Hepadnaviridae, Retroviridae, Reoviridae, Paramyxoviridae, Rhabdoviridae, Filoviridae, Orthomyxoviridae, Bunyaviridae, Arenaviridae, Bornaviridae, Picomaviridae, Caliciviridae, Astroviridae, Coronaviridae, Togaviridae, and Flaviviridae.

117. The method of claim 108 wherein the two or more viruses are selected from a group consisting of hepatitis C virus, yellow fever virus, respiratory syncytial virus, Sindbis virus, poliovirus, Japanese encephalitis virus, hepatitis B virus, human papilloma virus, herpes simplex virus type 1, Epstein-Barr virus, adeno-associated virus, Venezuela encephalitis virus, rubella, coxsackivirus, enterovirus, hepatitis A virus, Dengue fever virus, West Nile virus, tick-borne encephalitis virus, astrovirus, rabies virus, influenza virus A, influenza virus B, measles, mumps, Ebola virus, Marburg virus, La Crosse virus, California encephalitis virus, Hantaan virus, Crimean-Congo virus, Rift Valley fever, Lassa fever, Argentine hemorrhagic fever virus, Bolivian hemorrhagic fever virus, Colorado tick fever, JC virus, BK virus, herpes simplex virus type two, human cytomegalovirus, varicella-zoster virus, human herpes simplex virus type six, human herpes virus type seven, human herpes virus type eight, human adenovirus, HIV-1, HIV-2, HTLV-1, HTLV-2, and human parvovirus.
